(19) United States
(12) Patent Application Publication
(10) Pub. No.: US 2021/0198223 A1
(43) Pub. Date: Jul. 1, 2021

(54) METHODS AND COMPOSITIONS RELATING TO ULTRAPURE 5-(1,1-DIMETHYLHEPTYL)-RESORCINOL

(71) Applicant: Corbus Pharmaceuticals, Inc., Norwood, MA (US)

(72) Inventors: Valdas JURKAUSKAS, Cambridge, MA (US); Kristos Adrian MOSHOS, Watertown, MA (US); Clifton D. LEIGH, Somerville, MA (US); Feng SHI, Shanghai (CN); Xiaofeng CHEN, Shanghai (CN); Chengfu XIE, Shanghai (CN)

(21) Appl. No.: 16/756,944

(22) PCT Filed: Oct. 19, 2018
(86) PCT No.: PCT/US2018/056641

§ 371 Date: Apr. 17, 2020

Related U.S. Application Data
(60) Provisional application No. 62/577,868, filed on Oct. 27, 2017.

(30) Foreign Application Priority Data
Oct. 20, 2017 (CN) 201710984500.7

(51) Int. Cl. C07D 311/78; C07C 41/30

(52) U.S. Cl. CPC C07D 311/78; C07C 43/23; C07C 41/30

(57) ABSTRACT

The invention provides methods and compositions relating to an ultrapure formulation of 5-(1,1-dimethylheptyl)-resorcinol (ultrapure DMHR). The invention features methods for making ultrapure DMHR, including methods that minimize the production of unwanted side products (e.g., the production of homologous alkyl-chain impurities). The invention also features methods of making cannabinoids, such as (6aR,10aR)-1-hydroxy-6,6-dimethyl-3-(2-methyl-2-octanyl)-6a,7,10,10a-tetrahydro-6H-benzo[c]chromene-9-carboxylic acid (ajulemic acid), using ultrapure DMHR, including methods that minimize the production of unwanted side products (e.g., the production of homologous alkyl-chain impurities) in the resulting cannabinoid preparation.

### BACKGROUND OF THE INVENTION

Tetrahydrocannabinol (THC) is the major psychoactive constituent of marijuana. In addition to mood-altering effects, THC has been reported to exhibit other activities, some of which may have therapeutic value. The potential therapeutic value of THC has led to a search for related compounds which minimize the psychoactive effects, while retaining the activities of potential medicinal value.

One such related cannabinoid is (6aR,10aR)-1-hydroxy-6,6-dimethyl-3-(2-methyl-2-octanyl)-6a,7,10,10a-tetrahydro-6H-benzo[c]chromene-9-carboxylic acid (also known as ajulemic acid, AJA, JBT-101, resunab, anabasum, or lenabasum). Ajulemic acid has been investigated for its potential therapeutic benefits in a number of diseases, including fibrotic diseases and inflammatory diseases, for which there is a need for new therapies with improved safety and efficacy profiles.

Drugs currently used to treat chronic, serious diseases with chronic inflammation and fibrosis are divided broadly into several groups: non-steroidal anti-inflammatory drugs (NSAIDS), anti-malarial agents, systemic corticosteroids, and immunosuppressive agents.

The potency of NSAIDs can be too limited to control disease activity, and patients may receive additional treatment with anti-malarial drugs, systemic corticosteroids or immunosuppressive agents. Anti-malarial therapy can also be used as a baseline treatment for chronic inflammation in certain autoimmune diseases, Anti-malarial therapy frequently is ineffective in controlling chronic, serious inflammation, or can cause drug reactions. Antimalarial-refractory disease is then treated with systemic therapies that may additionally cause toxicity, including systemic corticosteroids and immunosuppressive agents.

Systemic corticosteroids are commonly prescribed for treatment of chronic, serious diseases characterized by chronic inflammation and fibrosis, such as cystic fibrosis, systemic sclerosis, and dermatomyositis. Chronic corticosteroid use can be limited by toxicities that include growth retardation, iatrogenic Cushings's Disease, hypertension, high glucose levels/diabetes, obesity, brittle bones, osteoporosis, aseptic necrosis of bone, immunosuppression, increased infection, glaucoma, depression, and psychosis. Thus, safer yet potent alternatives to steroids have long been sought.

Multiple other immunosuppressive drugs can be used to treat chronic, serious, inflammatory diseases, to achieve disease control and to reduce or avoid the need for corticosteroids. These include biological agents, such as monoclonal antibodies or fusion proteins, which target a very specific molecule in a key disease pathway. These drugs can have a number of disadvantages, including that the drugs must be administered parenterally and they are associated with increased incidence of malignancy and infection. Non-biologic immunosuppressive agents that can be used to treat chronic, serious inflammation include methotrexate, mycophenolate, leflunomide, cyclophosphamide, and azathioprine, among others. Intravenous immunoglobulin is used occasionally to treat refractory chronic, serious inflammatory diseases.

Treatment with cannabinoids, such as ajulemic acid, may offer a new therapeutic modality for diseases, including fibrotic diseases and inflammatory diseases. In particular, ajulemic acid may provide an improved safety profile over available treatment options for such diseases.

Development of ajulemic acid as a therapeutic has been limited, in part, by challenges associated with production of ajulemic acid with sufficient purity. Ajulemic acid may be produced by coupling para-mentha-2,8-dien-1-ol (PMD) with 5-(1,1-dimethylheptyl)-resorcinol (DMHR) as described in, for example, U.S. Patent Publication No. 2015/0141501. Homologous alkyl-chain impurities produced during the synthesis of DMHR may be carried through to later steps in the ajulemic acid synthesis, resulting in homologous alkyl-chain impurities in the ajulemic acid, which may alter the pharmacology and toxicology of the resulting preparation of ajulemic acid. This presents a particular challenge where the separation of homologous alkyl-chain impurities is costly and time consuming, and where on a large commercial-scale synthesis of DMHR or ajulemic acid, purification methods, such as high-performance liquid chromatography, may be impractical.

### SUMMARY OF THE INVENTION

The invention provides methods and compositions relating to an ultrapure preparation of compound (4) (below), and the use of compound (4) in the synthesis of an ultrapure preparation of 5-(1,1-dimethylheptyl)-resorcinol (ultrapure DMHR) and cannabinoids, such as ajulemic acid. The invention features methods for making ultrapure DMHR, including methods that minimize the production of unwanted side products (e.g., the production of homologous alkyl-chain impurities). The invention also features methods of making cannabinoids, such as (6aR,10aR)-1-hydroxy-6,6-dimethyl-3-(2-methyl-2-octanyl)-6a,7,10,10a-tetrahydro-6H-benzo[c]chromene-9-carboxylic acid (ajulemic acid), using ultrapure DMHR, including methods that minimize the production of unwanted side products (e.g., the production of homologous alkyl-chain impurities) in the resulting cannabinoid (e.g., ajulemic acid).

In a first aspect, the invention features a method of making a compound (4):

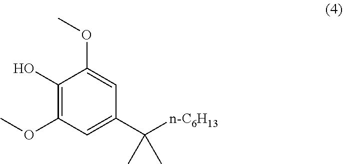

wherein the method includes the step of (i) adding a solution containing 1 molar equivalent of 2-methyloctan-2-ol to an acidic solution containing at least 1.1 molar equivalents of 1,3-dimethoxy-2-hydroxybenzene to form a mixture of compounds of formula (I):

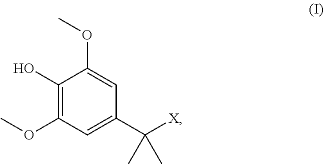

wherein X is a linear or branched C1-C10 alkyl, and wherein the mixture includes at least 98.0% (e.g., at least 98.5%, at least 99.0%, at least 99.5%, at least 99.8% or at least 99.9%) compound (4) and less than 2.0% (e.g., less than 1%, less than 0.5%, less than 0.2%, or less than 0.1%) compounds of formula ( ) in which X is a linear or branched C1-C10 alkyl other than n-C₆H₁₃; wherein the solution containing 2-methyloctan-2-ol is added to the acidic solution containing 1,3-dimethoxy-2-hydroxybenzene over the course of at least 1 hour; or wherein step (i) is performed at a temperature of between 20° C. and 55° C. (e.g., between 20° C. and 30° C., between 30° C. and 40° C., between 40° C. and 50° C., or between 50° C. and 55° C.).

In some embodiments, step (i) includes adding a solution containing 1 molar equivalent of 2-methyloctan-2-ol to an acidic solution containing at least 1.2 molar equivalents, 1.3 molar equivalents, or 1.4 molar equivalents of 1,3-dimethoxy-2-hydroxybenzene.

In some embodiments, the solution containing 2-methyloctan-2-ol is added to the acidic solution containing 1,3-dimethoxy-2-hydroxybenzene over the course of at least 2 hours, at least 4 hours, at least 6 hours, or more.

In some embodiments, step (i) is quenched after 1 hour, 2 hours, 3 hours, 4 hours, 5 hours, or more.

In some embodiments, step (i) is quenched before 75%, before 70%, before 65%, before 60%, before 55%, before 50%, before 45%, or before 40% of the 2-methyloctan-2-ol is converted into compound (4).

In some embodiments, step (i) is performed at a temperature of between 20° C. and 50° C. (e.g., between 20° C. and 30° C., between 30° C. and 40° C., between 40° C. and 50° C., or between 50° C. and 55° C.).

In some embodiments, the mixture includes at least 98.0% (e.g., at least 98.5%, at least 99.0%, at least 99.5%, at least 99.8% or at least 99.9%) compound (4).

In some embodiments, the mixture includes less than 1.5%, less than 1.0%, less than 0.75%, less than 0.5%, or less than 0.25% compounds of formula (I) in which X is a linear or branched C1-C10 alkyl other than n-C₆H₁₃.

In some embodiments, the mixture includes less than 1.0%, less than 0.75%, less than 0.50%, less than 0.25%, less than 0.20%, less than 0.15%, less than 0.10%, or less than 0.05% compounds of formula (I) in which X is n-C₅H₁₁ or —CH₂CH(CH₃)CH₂CH₂CH₂CH₃.

In some embodiments, step (i) includes producing greater than 0.5 kg, greater than 2 kg, greater than 5 kg, or greater than 10 kg of compound (4).

In some embodiments, the method further includes subjecting compound (4) to hydrogenation and demethylation to produce 5-(1,1-dimethylheptyl)-resorcinol (DMHR):

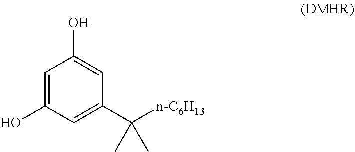

in a mixture of compounds of formula (II):

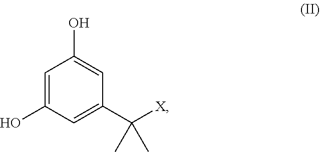

wherein X is a linear or branched C1-C10 alkyl, and wherein the mixture includes at least 98.0% (e.g., at least 98.5%, at least 99.0%, at least 99.5%, at least 99.8% or at least 99.9%) DMHR and less than 2.0% (e.g., less than 1%, less than 0.5%, less than 0.2%, or less than 0.1%) compounds of formula (II) in which X is a linear or branched C1-C10 alkyl other than n-C₆H₁₃.

In some embodiments, the mixture includes less than 1.5%, less than 1.0%, less than 0.75%, less than 0.5%, or less than 0.25% compounds of formula (II) in which X is a linear or branched C1-C10 alkyl other than n-C₆H₁₃.

In some embodiments, the mixture includes less than 1.0%, less than 0.75%, less than 0.50%, less than 0.25%, less than 0.20%, less than 0.15%, less than 0.10%, or less than 0.05% compounds of formula (II) in which X is n-C₅H₁₁ or —CH₂CH(CH₃)CH₂CH₂CH₂CH₃.

In some embodiments, hydrogenation and demethylation to produce DMHR includes producing greater than 0.5 kg, greater than 2 kg, greater than 5 kg, or greater than 10 kg of DMHR.

In some embodiments, the method further includes reacting para-mentha-2,8-dien-1-ol (PMD) and the DMHR to form compound (12):

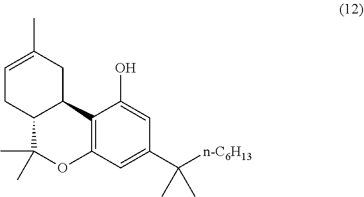

in a mixture of compounds of formula (III):

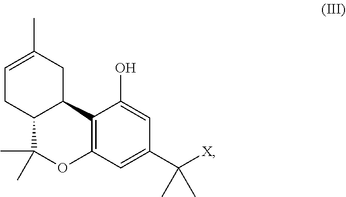

wherein X is a linear or branched C1-C10 alkyl, and wherein the mixture includes at least 98.0% (e.g., at least 98.5%, at least 99.0%, at least 99.5%, at least 99.8% or at least 99.9%) compound (12) and less than 2.0% (e.g., less than 1%, less than 0.5%, less than 0.2%, or less than 0.1%) compounds of formula (III) in which X is a linear or branched C1-C10 alkyl other than n-C₆H₁₃.

In some embodiments, the mixture includes less than 1.5%, less tan 1.0%, less Man 0.75%, less than 0.5%, or less than 0.25% compounds of formula (III) in which X is a linear or branched C1-C10 alkyl other than n-C₆H₁₃.

In some embodiments, the mixture includes less than 1.0%, less than 0.75%, less than 0.50%, less than 0.25%, less than 0.20%, less than 0.15%, less than 0.10%, or less than 0.05% compounds of formula (III) in which X is n-C₅H₁₁ or —CH₂CH(CH₃)CH₂CH₂CH₂CH₃.

In some embodiments, reacting PMD and DMHR to produce compound (12) includes producing greater than 0.5 kg, greater than 2 kg, greater than 5 kg, or greater than 10 kg of compound (12).

In some embodiments, the method further includes oxidizing compound (12) to form ajulemic acid (AJA):

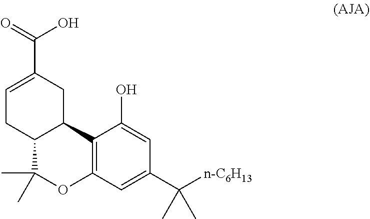

in a mixture of compounds of formula (IV):

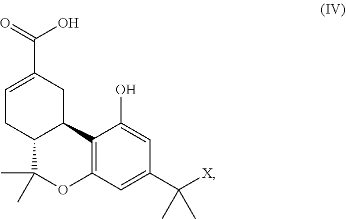

wherein X is a linear or branched C1-C10 alkyl, and wherein the mixture includes at least 98.0% (e.g., at least 98.5%, at least 99.0%, at least 99.5%, at least 99.8% or at least 99.9%) AJA and less than 2.0% (e.g., less than 1%, less than 0.5%, less than 0.2%, or less than 0.1%) compounds of formula (IV) in which X is a linear or branched C1-C10 alkyl other than n-C₆H₁₃.

In some embodiments, the mixture includes less than 1.5%, less than 1.0%, less than 0.75%, less than 0.5%, or less than 0.25% compounds of formula (IV) in which X is a linear or branched C1-C10 alkyl other than n-C₆H₁₃.

In some embodiments, the mixture includes less than 1.0%, less than 0.75%, less than 0.50%, less than 0.25%, less than 0.20%, less than 0.15%, less than 0.10%, or less than 0.05% compounds of formula (IV) in which X is n-C₅H₁₁ or —CH₂CH(CH₃)CH₂CH₂CH₂CH₃.

In some embodiments, oxidizing compound (12) to form ajulemic acid includes producing greater than 0.5 kg, greater than 2 kg, greater than 5 kg, or greater than 10 kg of compound (12).

In another aspect, the invention features a pharmaceutical composition including ajulemic acid, or a salt thereof, produced according to any of the methods described herein, and a pharmaceutically acceptable excipient.

In some embodiments, the invention features a method of treating an inflammatory condition in a subject in need thereof, wherein the method includes administering to the subject a pharmaceutical composition including ajulemic acid, or a salt thereof, produced according to any of the methods described herein, and a pharmaceutically acceptable excipient in an amount sufficient to treat the condition.

In some embodiments, the invention features a method of treating a fibrotic condition in a subject in need thereof, wherein the method includes administering to the subject a pharmaceutical composition including ajulemic acid, or a salt thereof, produced according to any of the methods described herein, and a pharmaceutically acceptable excipient in an amount sufficient to treat the condition.

In some embodiments, the invention features a method wherein ultrapure 5-(1,1-dimethylheptyl)resorcinol (DMHR) is further reacted in a synthesis to produce a cannabinoid (e.g., ajulemic acid or any one of Compounds 20-125).

In some embodiments, the invention features a pharmaceutical composition comprising a cannabinoid (e.g., ajulemic acid or any one of Compounds 20-125), or a salt thereof, produced according any of the methods described herein and a pharmaceutically acceptable excipient.

### Definitions

To facilitate the understanding of this invention, a number of terms are defined below. Terms defined herein have meanings as commonly understood by a person of ordinary skill in the areas relevant to the invention. Terms such as “a”, “an,” and “the” are not intended to refer to only a singular entity, but include the general class of which a specific example may be used for illustration. The terminology herein is used to describe specific embodiments of the invention, but their usage does not limit the invention, except as outlined in the claims.

As used herein, the term “about” refers to a value that is within 10% above or below the value being described.

As used herein, any values provided in a range of values include both the upper and lower bounds, and any values contained within the upper and lower bounds.

As used herein, the term “treat” or “treatment” includes administration of a compound, e.g., by any route, e.g., orally, topically, or by inhalation to a subject. The compound can be administered alone or in combination with one or more additional compounds. Treatments may be sequential, with the present compound being administered before or after the administration of other agents. Alternatively, compounds may be administered concurrently. The subject, e.g., a patient, can be one having a disorder (e.g., a disorder as described herein), a symptom of a disorder, or a predisposition toward a disorder. Treatment is not limited to curing or complete healing, but can result in one or more of alleviating, relieving, altering, partially remedying, ameliorating, improving or affecting the disorder, reducing one or more symptoms of the disorder or the predisposition toward the disorder. In an embodiment the treatment (at least partially) alleviates or relieves symptoms related to a fibrotic disease. In an embodiment the treatment (at least partially) alleviates or relieves symptoms related to an inflammatory disease. In one embodiment, the treatment reduces at least one symptom of the disorder or delays onset of at least one symptom of the disorder. The effect is beyond what is seen in the absence of treatment.

The term “pharmaceutical composition” refers to the combination of an active agent with an excipient, inert or active, making the composition especially suitable for diagnostic or therapeutic use in vivo or ex vivo. A “pharmaceutically acceptable excipient,” after administered to or upon a subject, does not cause undesirable physiological effects. The excipient in the pharmaceutical composition must be “acceptable” also in the sense that it is compatible with the active ingredient and can be capable of stabilizing it. One or more solubilizing agents can be utilized as pharmaceutical excipients for delivery of an active compound. Examples of a pharmaceutically acceptable excipients include, but are not limited to, biocompatible vehicles, adjuvants, additives, and diluents to achieve a composition usable as a dosage form. Examples of other excipients include colloidal silicon oxide, magnesium stearate, cellulose, and sodium lauryl sulfate.

As used herein, the term “carrier” refers to a diluent, adjuvant, excipient, or vehicle with which the active compound is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents can be used. When administered to a subject, the pharmaceutically acceptable vehicles are preferably sterile. Water can be the vehicle when the active compound is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, sodium stearate, glycerol monostearate, talc, sodium chloride, glycerol, propylene glycol, water, and ethanol. The present compositions, if desired, can also contain minor amounts of wetting or emulsifying agents, or pH buffering agents.

The term “alkyl” as used herein, include straight-chain and branched-chain monovalent substituents containing only C and H. In some embodiments, the alkyl group may contain, e.g., 1-10. 4-10, 4-8, 5-10, 4-7, or 7-10 carbon atoms (e.g., C1-C10, C4-C8, C5-C10, C4-C7, or C7-C10). Examples include, but are not limited to, isobutyl, sec-butyl, n-pentyl (e.g., n-C₅H₁₁), n-hexyl (e.g. n-C₆H₁₃), n-heptyl, —CH2CH(CH3)CH2CH2CH2CH3, and n-octyl, among others.

The term “ultrapure DMHR,” as used herein, refers to 5-(1,1-dimethylheptyl)-resorcinol (DMHR) which has been produced according to any of the methods described herein. In some embodiments, ultrapure DMHR may be produced according the synthetic scheme of FIG. 2. In some embodiments, ultrapure DMHR may be produced according to the methods described in Example 5. In some embodiments, ultrapure DMHR includes a mixture of compounds of formula (II):

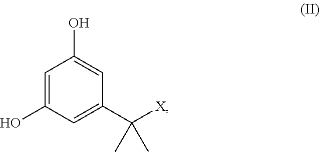

wherein X is a linear or branched C1-C10 alkyl, and wherein the mixture includes at least 98.0% (e.g., at least 98.5%, at least 99.0%, at least 99.5%, at least 99.8% or at least 99.9%) DMHR and less than 2.0% (e.g., less than 1%, less than 0.5%, less than 0.2%, or less than 0.1%) compounas of formula (II) in which X is a linear or branched C1-C10 alkyl other than n-C₆H₁₃.

### BRIEF DESCRIPTION OF THE DRAWINGS

FIG. 1 is scheme showing a synthetic route for the production of 5-(1,1-dimethylheptyl)-resorcinol (DMHR).

FIG. 2 is scheme showing a synthetic route for the production of ultrapure 5-(1,1-dimethylheptyl)-resorcinol (DMHR).

FIG. 3 is a gas chromatograph showing the comparison of the 2-octanone starting material used for the synthesis of DMHR to reference samples of homologous alkyl-chain impurities: n-heptanone, 3-octanone, and n-nonanone. No significant amount of these homologous alkyl-chain impurities were observed to be present in the 2-octanone starting material.

FIG. 4A is an ¹H NMR spectra showing the presence of a linear alkyl-chain impurity, Compound (7) (e.g., impurity having one carbon less) having a relative retention time (RRT) of 0.96 produced by Step 2 of the production of DMHR.

FIG. 4B is mass spectrometry trace showing the characterization of the linear alkyl chain impurity, Compound (7) (e.g., impurity having one carbon less) of FIG. 4A.

FIG. 5A is an ¹H NMR spectra showing the presence of a branched alkyl-chain impurity, Compound (8) (e.g., impurity having one carbon more) having a relative retention time (RRT) of 1.03 produced by Step 2 in the production of DMHR.

FIG. 5B is a mass spectrometry trace showing the characterization of the branched alkyl-chain impurity, Compound (8) (e.g., impurity having one carbon more) of FIG. 5A.

FIG. 6 is a scheme showing the proposed mechanism for the formation of homologous impurities in Step 2 in the production of DMHR.

FIG. 7 is a graph showing decreased production of homologous alkyl-chain impurities, Compounds (7) and (8) (e.g., one carbon less impurity and one carbon more impurity) as a result of slow addition (e.g., 6 hours addition) of 1,3-dimethoxy-2-hydroxybenzene in Step 2 of the production of DMHR as compared to the original conditions (e.g., rapid addition over 1 hour or less).

FIG. 8 is a graph showing decreased production of homologous alkyl-chain impurities, Compounds (7) and (8) (e.g., one carbon less impurity and one carbon more impurity) as a result of a molar excess of 1,3-dimethoxy-2-hydroxybenzene over 2-methyloctan-2-ol (e.g., 1.2:1 1,3-dimethoxy-2-hydroxybenzene:2-methyloctan-2-ol) in Step 2 of the production of DMHR as compared to the original conditions (e.g., 1:1 1,3-dimethoxy-2-hydroxybenzene: 2-methyloctan-2-ol).

FIG. 9 is a graph showing the production of product, Compound (4), and homologous alkyl-chain impurities, Compounds (7) and (8) (e.g., one carbon less and one carbon more impurities) over time during Step 2 of the synthesis of DMHR. The rate of production of the product and impurities shows that quenching the reaction at a time point prior to completion of the reaction may increase the ratio of product to alkyl-chain impurities.

FIG. 10 is a set of graphs showing that reduction in reaction temperature of Step 2 of the synthesis of DMHR may decrease the production of homologous alkyl-chain impurities, Compounds (7) and (8) (e.g., one carbon less and once carbon more impurities).

FIG. 11 is a graph showing that prolonged carbocation residence time increases production of homologous alkyl-chain impurities, Compounds (7) and (8) (e.g., one carbon less and once carbon more impurities). 2-methyloctan-2-ol was combined with MsOH for 6 hours prior to addition of 1,3-dimethoxy-2-hydroxybenzene resulting in an increase in the production of impurities, and a decrease in conversion to the desired product. Accordingly, contact time between 2-methylheptan-2-ol and MsOH prior to addition of 1,3-dimethoxy-2-hydroxybenzene should be minimized.

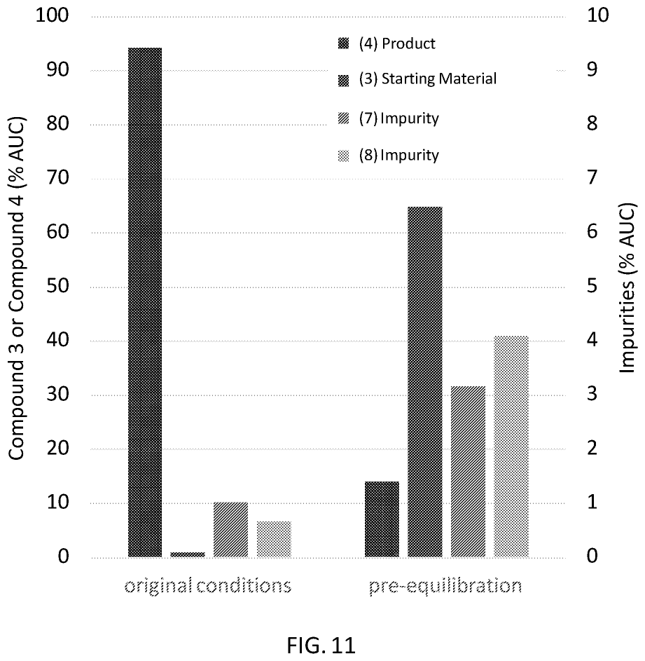

FIGS. 12A-B are a set of graphs showing decreased production of homologous alkyl-chain impurities (e.g., one carbon less impurity and one carbon more impurity) as a result of the optimized reaction conditions of Step 2 of the synthesis of DMHR, as described herein (e.g., slow addition of 1,3-dimethoxy-2-hydroxybenzene and use of excess 1,3-dimethoxy-2-hydroxybenzene). FIG. 12A shows the resulting increase in purity of Compound (4). FIG. 12B shows the corresponding increase in purity carried through in the synthesis of DMHR.

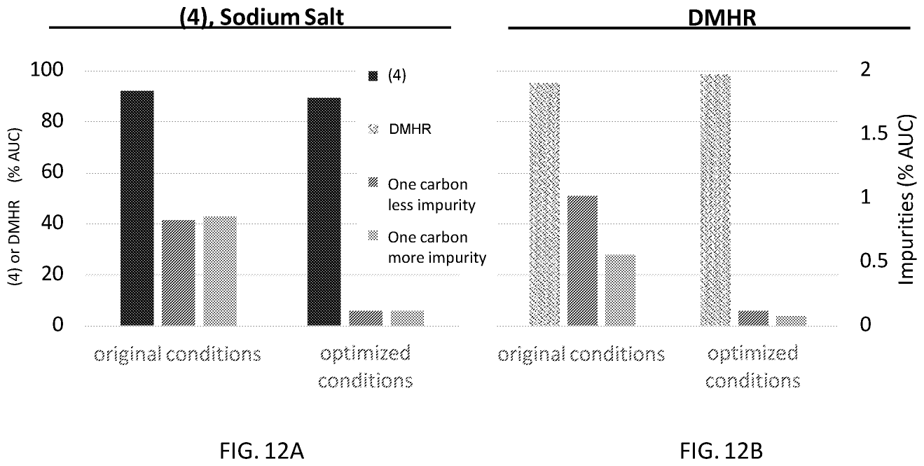

FIG. 13 is a synthetic scheme for the production of ajulemic acid, beginning with the coupling of PMD and DMHR.

FIG. 14 shows the increase in purity and decrease in homologous alkyl-chain impurities in ajulemic acid, such as Compound (9), produced with ultrapure DMHR which was synthesized according to the optimized protocols described herein.

### DETAILED DESCRIPTION OF THE INVENTION

In general, the invention features methods and compositions relating to an ultrapure formulation of 5-(1,1-dimethylheptyl)-resorcinol (ultrapure DMHR). The invention features methods for making ultrapure DMHR, including methods that minimize the production of unwanted side products (e.g., the production of homologous alkyl-chain impurities). The invention also features methods of making cannabinoids, such as (6aR,10aR)-1-hydroxy-6,6-dimethyl-3-(2-methyl-2-octanyl)-6a,7,10,10a-tetrahydro-6H-benzo[c]chromene-9-carboxylic acid (ajulemic acid), using ultrapure DMHR, including methods that minimize the production of unwanted side products (e.g., the production of homologous alkyl-chain impurities) in the resulting cannabinoid (e.g., ajulemic acid).

### Homologous Alkyl-Chain Impurities

The methods of the invention may be used to minimize the productions of homologous alkyl-chain impurities in the production of DMHR, or cannabinoids such as ajulemic acid, or any intermediate compound produced in the synthesis of DMHR or cannabinoids such as ajulemic acid (e.g., according to the synthetic methods described herein). Homologous alkyl-chain impurities include any compound belonging to a series of compounds (e.g., Formulas (I)-(IV), as described herein), where the impurity differs from the desired compound (e.g., DMHR or ajulemic acid) such that an alkyl chain of the compound includes a different length (e.g., more or less carbons) than the alkyl chain of the desired compound or the alkyl chain is an isomer of the alkyl chain of the desired compound.

Homologous alkyl-chain impurities may be produced by the coupling of 2-methyloctan-2-ol to 1,3-dimethoxy-2-hydroxybenzene to produce Compound (4):

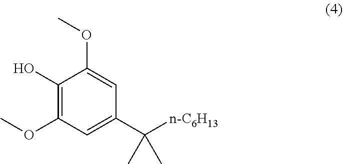

The coupling to produce Compound (4), may further produce homologous alkyl-chain impurities having a structure according to Formula (I):

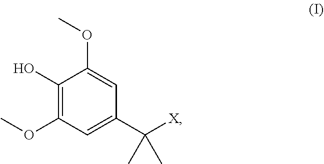

wherein X is a linear or branched C1-C10 alkyl other than n-C₆H₁₃.

Homologous alkyl-chain impurities produced in the above coupling may be carried through the synthesis of DMHR, e.g., carried through hydrogenation and subsequent demethylation of Compound (4) to produce DMHR:

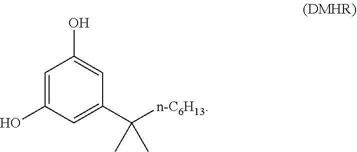

The production of DMHR may result in the production of homologous alkyl-chain impurities of DMHR having a structure according to Formula (II):

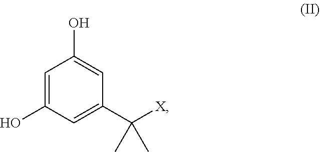

wherein X is a linear or branched C1-C10 alkyl other than n-C₆H₁₃.

Homologous alkyl-chain impurities may further be carried through the synthesis of ajulemic acid, beginning with DMHR as a starting material. For example, such impurities may be carried through the coupling of para-mentha-2,8-dien-1-ol (PMD) and DMHR to form Compound (12):

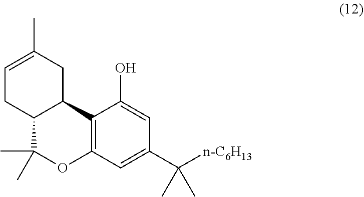

The production of Compound (12) may result in the production of homologous alkyl-chain impurities of Compound (12) having a structure according to Formula (III):

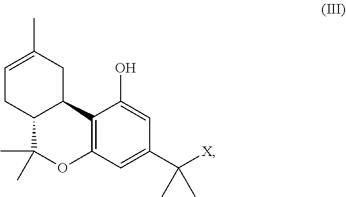

wherein X is a linear or branched C1-C10 alkyl other than n-C₆H₁₃.

Homologous alkyl-chain impurities may further be carried through the oxidation of Compound (12) to produce ajulemic acid (AJA):

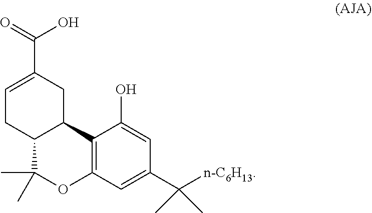

The production of ajulemic acid may result in the production of homologous alkyl-chain impurities of ajulemic acid having a structure according to Formula (IV):

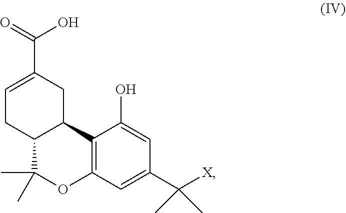

wherein X is a linear or branched C1-C10 alkyl other than n-C₆H₁₃.

### Preparation of Ultrapure DMHR

The present invention provides for a process for preparing DMHR, e.g., ultrapure DMHR, which minimizes the production of homologous alkyl-chain impurities.

In some embodiments, the DMHR produced by the methods described herein has a purity greater than about 98% (w/w), greater than about 99% (w/w), greater than about 99.1% (w/w), greater than about 99.2% (w/w), greater than about 99.3% (w/w), greater than about 99.4% (w/w), greater than about 99.5% (w/w) or greater than about 99.9% (w/w).

In some embodiments, the resulting DMHR has less than 5.0%, less than 4.0%, less than 3.0%, less than 2.0%, less than 1.0%, less than 0.5% or less than 0.1% compounds of formula (II), where formula (II) is

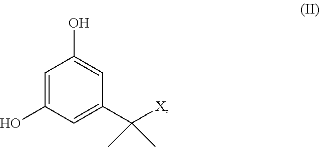

and
where X is a linear or branched C1-C10 alkyl other than n-C₆H₁₃,

In some embodiments, the DMHR preparation includes less than 1.0%, less than 0.75%, less than 0.50%, less than 0.25%, less than 0.20%, less than 0.15%, less than 0.10%, or less than 0.05% compounds of formula (II) in which X is n-C₅H₁₁ or —CH₂CH(CH₃)CH₂CH₂CH₂CH₃.

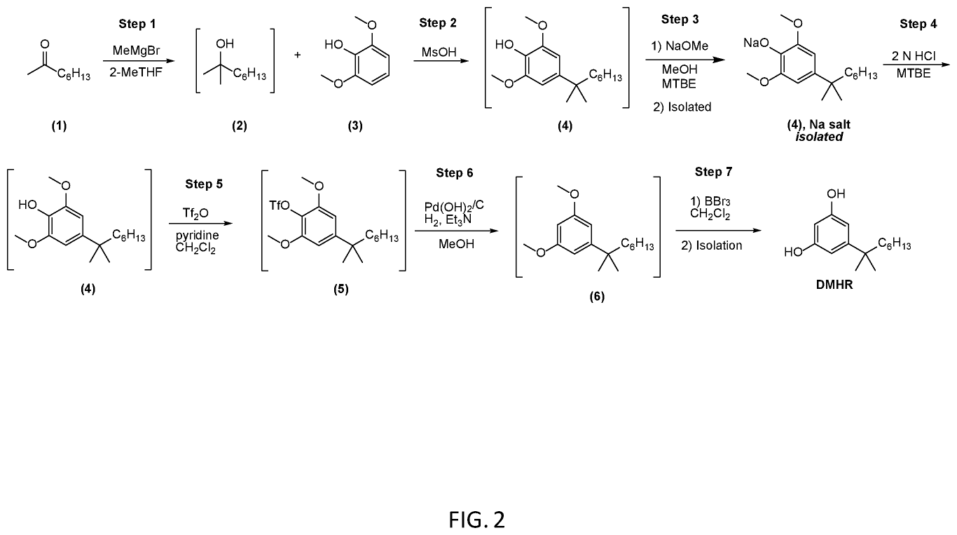

In some embodiments, production of ultrapure DMHR may be performed according to synthetic scheme of FIG. 2 and as described in Example 5.

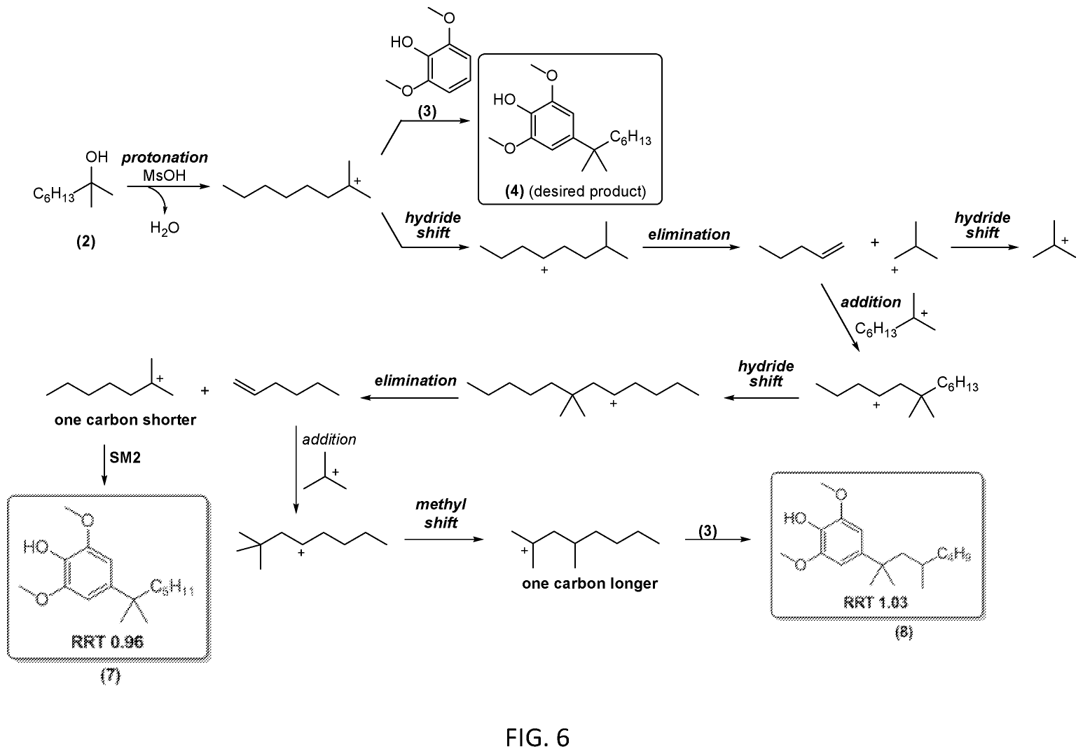

It had been previously assumed by those skilled in the art that control of homologous alkyl-chain impurities in DMHR is related to the quality of 2-octanone used in the preparation of DMHR. However, the present invention is based on the discovery that the coupling of 2-methyloctan-2-ol to 1,3-dimethoxy-2-hydroxybenzene to produce Compound (4) (e.g., Step 2 in the production of DMHR as described in Example 1) allows for the formation of homologous alkyl-chain impurities (e.g., homologous alkyl-chain impurities having the structure of Formula (I)). A proposed mechanism for the formation of homologous alky-chain impurities in the synthesis of DMHR is provided in FIG. 6. The inventors further determined that impurities generated in this step may be carried through in the production of DMHR (e.g., homologous alkyl-chain impurities having the structure of Formula (II)).

The present invention provides modifications to the production of DMHR that minimize the production of homologous alkyl-chain impurities.

### Modification A: Slow addition of 2-methylheptan-2-ol to 1,3-dimethoxy-2-hydroxybenzene

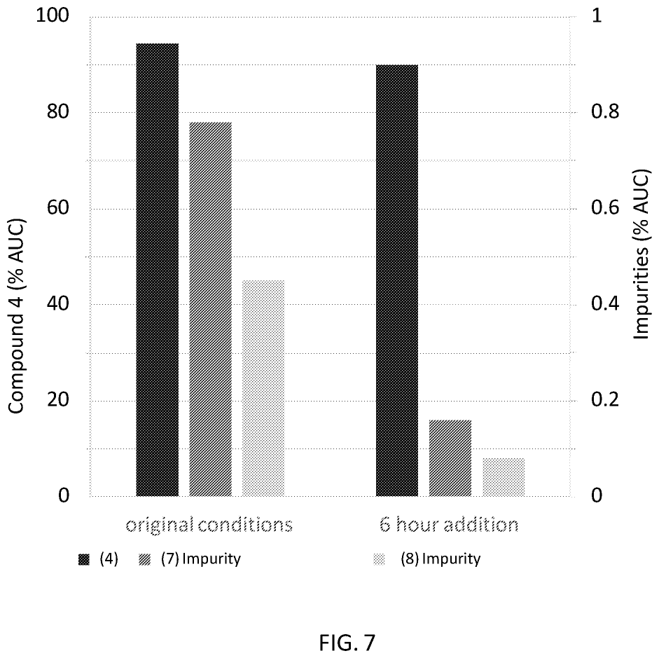

The inventors have discovered that the slow addition of the solution containing 2-methyloctan-2-ol to the acidic solution containing 1,3-dimethoxy-2-hydroxybenzene decreases the production of homologous alkyl-chain impurities in the production of Compound (4). As described in Example 5, a solution containing 2-methyloctan-2-ol was added to an acidic solution containing 1,3-dimethoxy-2-hydroxybenzene over 6 hours. The resulting decrease in the production of homologous alkyl chain impurities in the synthesis of Compound (4) is shown in FIG. 7.

Accordingly, in some embodiments, the solution containing 2-methylheptan-2-ol is added to the acidic solution containing 1,3-dimethoxy-2-hydroxybenzene over the course of at least 2 hours, at least 4 hours, at least 6 hours, or more.

### Modification B: Molar excess of 1,3-dimethoxy-2-hydroxybenzene over of 2-methyloctan-2-ol

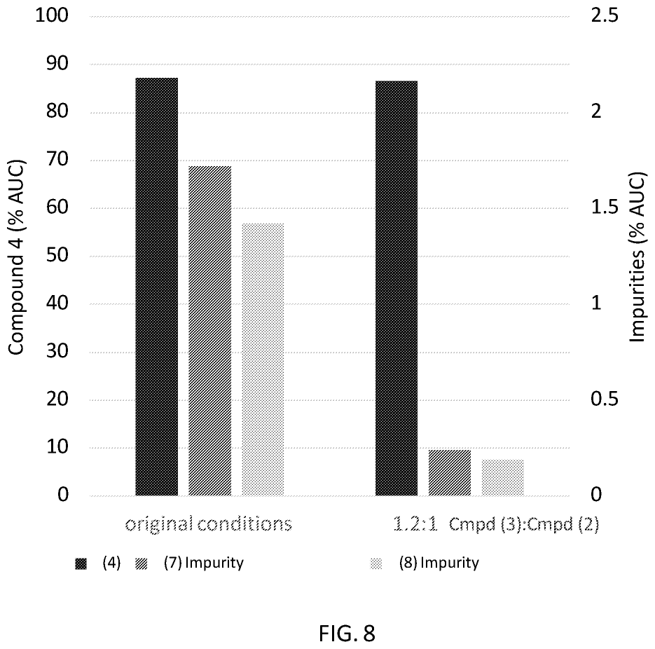

The inventors have further discovered that reacting a molar excess of 1,3-dimethoxy-2-hydroxybenzene with 2-methyloctan-2-ol to produce Compound (4) results in a decrease in the production of homologous impurities. As described in Example 5, the synthesis of Compound (4) was performed with a molar excess of 1.2 equivalents of 1,3-dimethoxy-2-hydroxybenzene over 1 equivalent of 2-methyloctan-2-ol. The resulting decrease in the production of homologous alkyl chain impurities in the synthesis of Compound (4) is shown in FIG. 8.

Accordingly, in some embodiments, the reaction of 2-methylheptan-2-ol and 1,3-dimethoxy-2-hydroxybenzene prior to produce Compound (4) includes a molar excess of 1,3-dimethoxy-2-hydroxybenzene over of 2-methyloctan-2-ol. In some embodiments, the molar excess is 1.1 equivalents 1,3-dimethoxy-2-hydroxybenzene to 1 equivalent 2-methyloctan-2-ol; 1.2 equivalents 1,3-dimethoxy-2-hydroxybenzene to 1 equivalent 2-methyloctan-2-ol; 1.3 equivalents 1,3-dimethoxy-2-hydroxybenzene to 1 equivalent 2-methyloctan-2-ol; 1.4 equivalents 1,3-dimethoxy-2-hydroxybenzene to 1 equivalent 2-methyloctan-2-ol; 1.5 equivalents 1,3-dimethoxy-2-hydroxybenzene to 1 equivalent 2-methyloctan-2-ol; 2.0 equivalents 1,3-dimethoxy-2-hydroxybenzene to 1 equivalent 2-methyloctan-2-ol; 3.0 equivalents 1,3-dimethoxy-2-hydroxybenzene to 1 equivalent 2-methyloctan-2-ol; or a greater excess of 1,3-dimethoxy-2-hydroxybenzene over of 2-methyloctan-2-ol.

### Modification C: Quenching of the reaction of 2-methyloctan-2-ol and 1,3-dimethoxy-2-hydroxybenzene

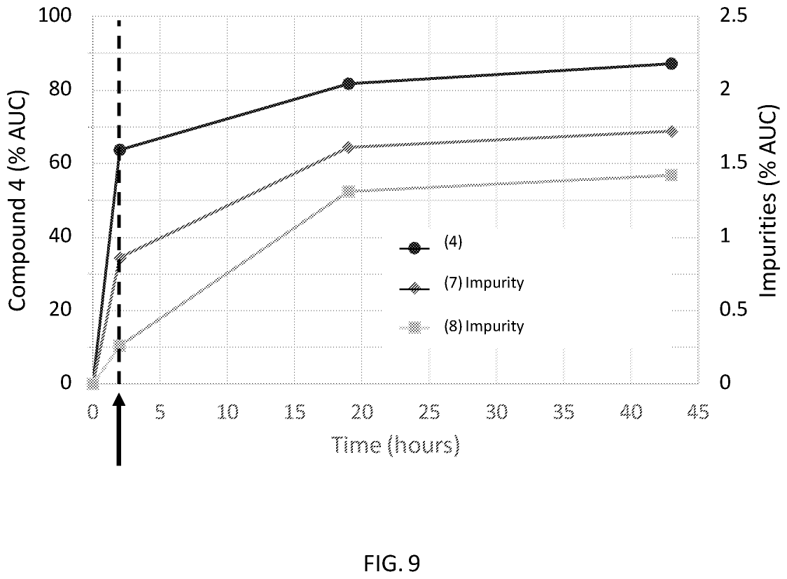

The inventors have discovered that quenching the reaction of 2-methyloctan-2-ol and 1,3-dimethoxy-2-hydroxybenzene prior to full conversion to Compound (4) decreases the production of homologous alkyl-chain impurities in the production of Compound (4). As described in Example 5, the rate of production of Compound (4) and corresponding homologous alkyl chain impurities was determined and the production of these compounds over time is shown in FIG. 9. The more rapid initial rate of production of Compound (4) as compared to the impurities suggests that quenching the reaction at a time point prior to completion of the reaction (e.g., after 2 hours or before 75% of the 2-methylheptan-2-ol is converted into Compound (4) may increase the ratio of product to alkyl-chain impurities.

Accordingly, in some embodiments, the reaction of 2-methyloctan-2-ol and 1,3-dimethoxy-2-hydroxybenzene is quenched after 1 hour, 2 hours, 3 hours, 4 hours, 5 hours, or more. In some embodiments, the reaction of 2-methyloctan-2-ol and 1,3-dimethoxy-2-hydroxybenzene is quenched before 75%, before 70%, before 65%, before 60%, before 55%, before 50%, before 45%, or before 40% of the 2-methyloctan-2-ol is converted into Compound (4).

### Modification D: Low temperature reaction of 2-methyloctan-2-ol and 1,3-dimethoxy-2-hydroxybenzene

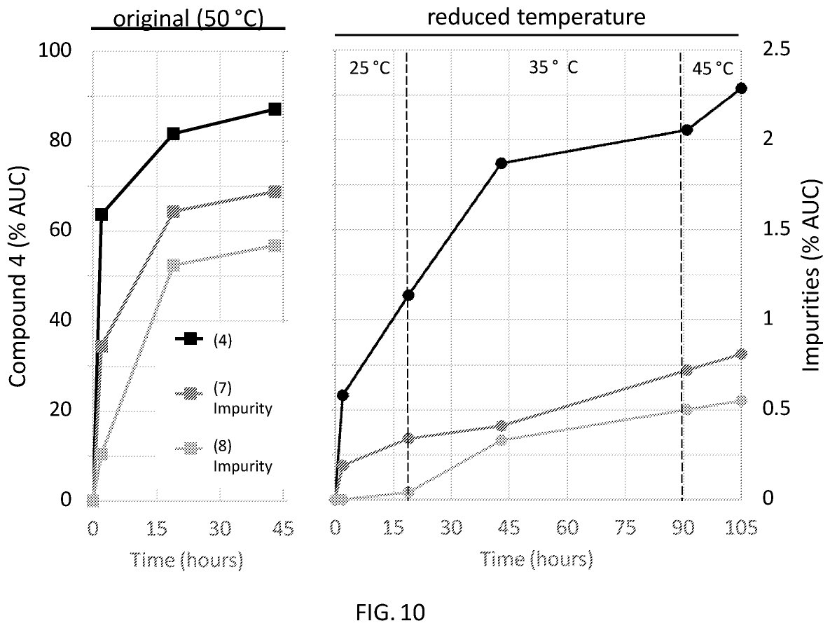

The inventors have discovered that decreasing the temperature of the reaction of 2-methyloctan-2-ol and 1,3-dimethoxy-2-hydroxybenzene to produce Compound (4) decreases the production of homologous alkyl-chain impurities. As described in Example 5, the reaction of 2-methyloctan-2-ol and 1,3-dimethoxy-2-hydroxybenzene to produce Compound 4 was performed at a reduced temperature of 25° C. for an initial period of time, followed by an increase to 35° C. for a second period of time, and 45° C. for a third period of time. This was compared to the reaction under the original conditions of 50° C. FIG. 10 shows that reduction in reaction temperature decreases the production of homologous alkyl-chain impurities.

Accordingly, in some embodiments, the reaction of 2-methyloctan-2-ol and 1,3-dimethoxy-2-hydroxybenzene is performed at a temperature of between 20° C. and 55° C. (e.g., between 20° C. and 30° C., between 30° C. and 40° C., between 40° C. and 50° C., or between 50° C. and 55° C.).

### Preparation of Ajulemic Acid Using Ultrapure DMHR

(6aR,10aR)-1-Hydroxy-6,6-dimethyl-3-(2-methyl-2-octanyl)-6a,7,10,10a-tetrahydro-6H-benzo[c]chromene-9-carboxylic acid (ajulemic acid) is a cannabinoid that is structurally related to THC, but which lacks the undesirable psychotropic effects associated with THC. As a result, ajulemic acid has been investigated for its potential therapeutic utility in a number of diseases including fibrotic diseases and inflammatory diseases.

The present invention provides for a process of preparing ajulemic acid using ultrapure DMHR as a starting material to minimize the production of unwanted homologous alkyl-chain impurities.

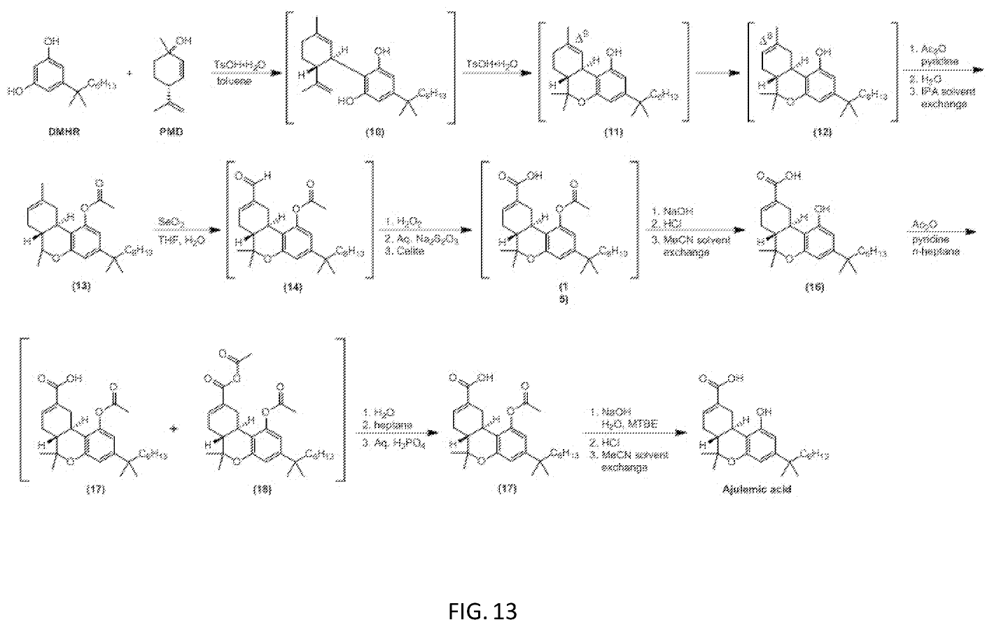

In some embodiments, the process for production of ajulemic acid using ultrapure DMHR may be performed according to the synthetic scheme provided in FIG. 13 and the methods described in Example 7.

In some embodiments, the ajulemic acid produced by the methods described herein has a purity greater than about 98% (w/w), greater than about 99% (w/w), greater than about 99.1% (w/w), greater than about 99.2% (w/w), greater than about 99.3% (w/w), greater than about 99.4% (w/w), greater than about 99.5% (w/w) or greater than about 99.9% (w/w).

In some embodiments, the ajulemic acid has less than 5.0%, less than 4.0%, less than 3.0%, less than 2.0%, less than 1.0%, less than 0.5% or less than 0.1% compounds of formula (IV), where formula (IV) is

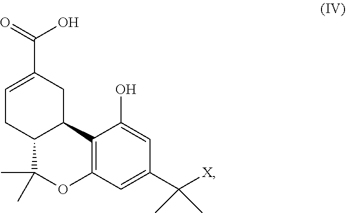

and
where X is a linear or branched C1-C10 alkyl other than n-C₆H₁₃,

In some embodiments, the ajulemic acid preparation includes less than 1.0%, less than 0.75%, less than 0.50%, less than 0.25%, less than 0.20%, less than 0.15%, less than 0.10%, or less than 0.05% compounds of formula (IV) in which X is n-C₅H₁₁ or —CH₂CH(CH₃)CH₂CH₂CH₂CH₃.

### Preparation of Cannabinoids Using Ultrapure DMHR

The present invention provides for methods for preparing cannabinoids using ultrapure DMHR as a starting material to minimize the production of unwanted homologous alkyl-chain impurities.

In some embodiments, the cannabinoid produced using ultrapure DMHR may be selected from ajulemic acid, a dimethylheptyl-cannabidiol (DMH-CBD) analog, or any dimethylheptyl-tetrahydrocannabinol (DMH-THC) analog.

Compounds 20-53 are exemplary DMH-CBD analogs of the invention, the structures of which are provided in Table 1. An exemplary synthetic protocol for the synthesis of DMH-CBD analogs is provided in, for example, Makriyannis, Alexandros et al. WO2014062965, which is incorporated herein by reference.

Compounds 54-125 are exemplary DMH-THC analogs of the invention, the structures of which are provided in Table 2. An exemplary synthetic protocol for the synthesis of DMH-THC analogs is provided in, for example, Mechoulam, R., Lander, N., Breuer, A.; Zahalka, J. Tetrahedron: Asymmetry 1(5):315-18, 1990, which is incorporated herein by reference.

TABLE 1

| | |
|---|---|
| 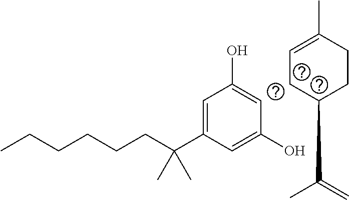 | Com- pound 20 |
| 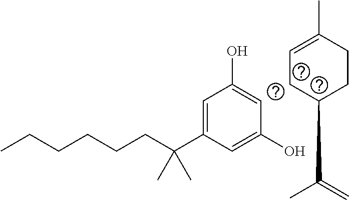 | Com- pound 21 |
| 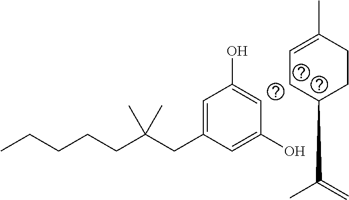 | Com- pound 22 |
| 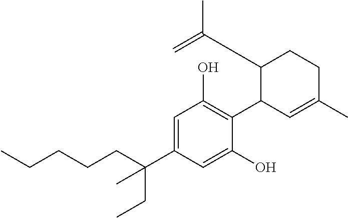 [SMILES: C=C(C)C1CCC(C)=CC1c1c(O)cc(C(C)(CC)CCCCC)cc1O] | Com- pound 23 |
| 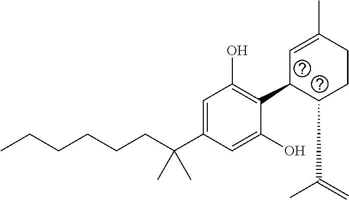 [SMILES: C=C(C)[C@H]1CCC(C)=C[C@@H]1c1c(O)cc(C(C)(C)CCCCCC)cc1O] | Com- pound 24 |
| 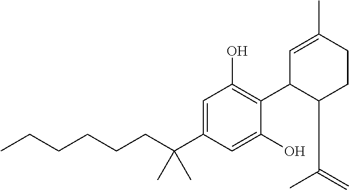 [SMILES: C=C(C)C1CCC(C)=CC1c1c(O)cc(C(C)(C)CCCCCC)cc1O] | Com- pound 25 |
| 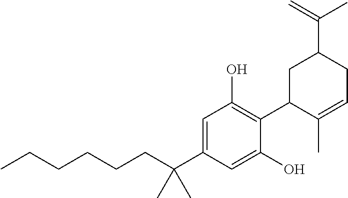 [SMILES: C=C(C)C1CC=C(C)C(c2c(O)cc(C(C)(C)CCCCCC)cc2O)C1] | Com- pound 26 |
| 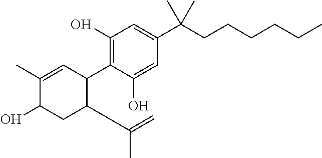 [SMILES: C=C(C)C1CC(O)C(C)=CC1c1c(O)cc(C(C)(C)CCCCCC)cc1O] | Com- pound 27 |
| 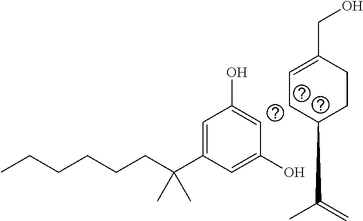 | Com- pound 28 |
| 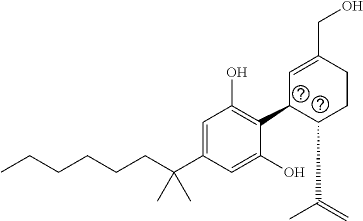 [SMILES: C=C(C)[C@H]1CCC(CO)=C[C@@H]1c1c(O)cc(C(C)(C)CCCCCC)cc1O] | Com- pound 29 |
| 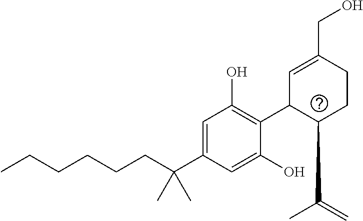 [SMILES: C=C(C)[C@@H]1CCC(CO)=CC1c1c(O)cc(C(C)(C)CCCCCC)cc1O] | Com- pound 30 |
| 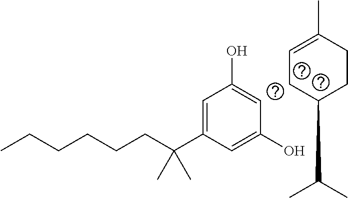 | Com- pound 31 |
| 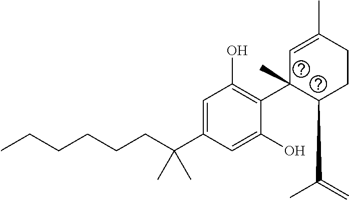 [SMILES: C=C(C)[C@@H]1CCC(C)=C[C@@]1(C)c1c(O)cc(C(C)(C)CCCCCC)cc1O] | Com- pound 32 |
| 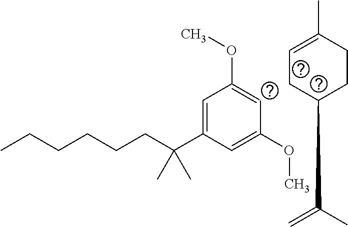 | Com- pound 33 |
| 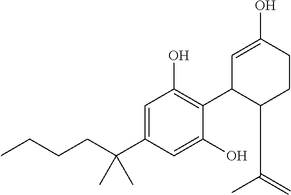 [SMILES: C=C(C)C1CCC(O)=CC1c1c(O)cc(C(C)(C)CCCC)cc1O] | Com- pound 34 |
| 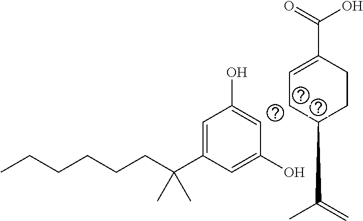 | Com- pound 35 |
| 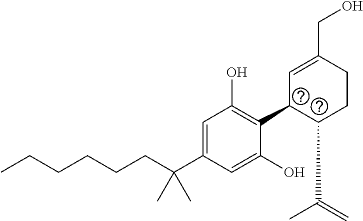 [SMILES: C=C(C)[C@H]1CCC(CO)=C[C@@H]1c1c(O)cc(C(C)(C)CCCCCC)cc1O] | Com- pound 36 |
| 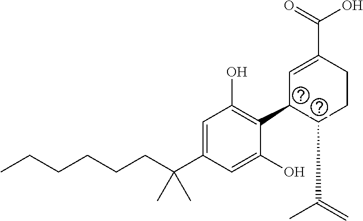 [SMILES: C=C(C)[C@H]1CCC(C(=O)O)=C[C@@H]1c1c(O)cc(C(C)(C)CCCCCC)cc1O] | Com- pound 37 |
| 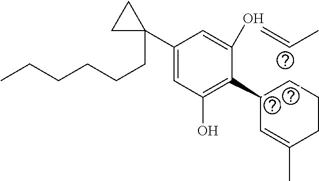 | Com- pound 38 |
| 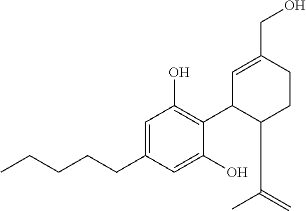 [SMILES: C=C(C)C1CCC(CO)=CC1c1c(O)cc(CCCCC)cc1O] | Com- pound 39 |
| 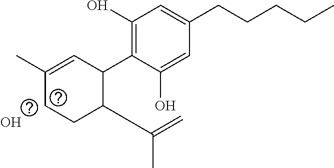 | Com- pound 40 |
| 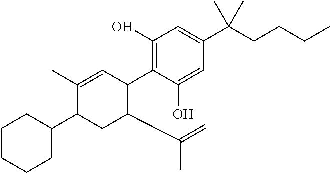 [SMILES: C=C(C)C1CC(C2CCCCC2)C(C)=CC1c1c(O)cc(C(C)(C)CCCC)cc1O] | Com- pound 41 |
| 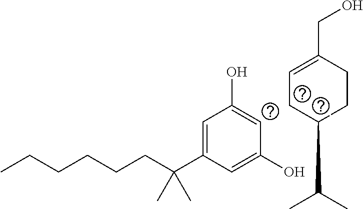 | Com- pound 42 |
| 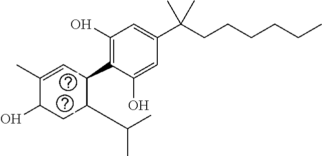 [SMILES: CCCCCCC(C)(C)c1cc(O)c([C@@H]2C=C(C)C(O)CC2C(C)C)c(O)c1] | Com- pound 43 |
| 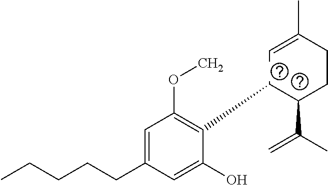 [SMILES: C=C(C)[C@@H]1CCC(C)=C[C@H]1c1c(O)cc(CCCCC)cc1OC] | Com- pound 44 |
| 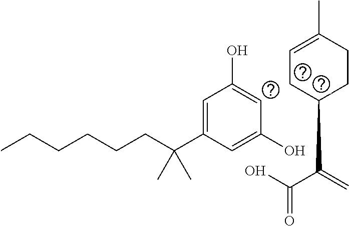 | Com- pound 45 |
| 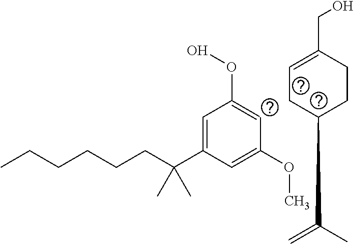 | Com- pound 46 |
| 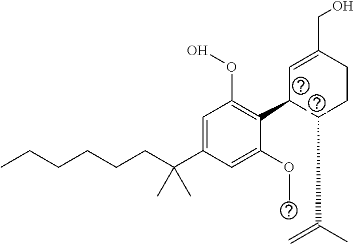 [SMILES: C=C(C)[C@H]1CCC(CO)=C[C@@H]1c1c(OC)cc(C(C)(C)CCCCCC)cc1OO] | Com- pound 47 |
| 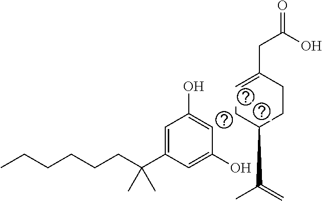 | Com- pound 48 |
| 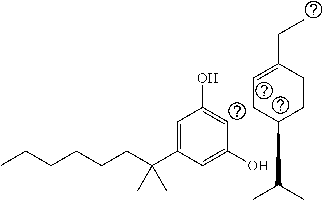 | Com- pound 49 |
| 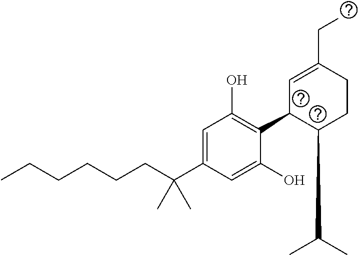 [SMILES: CCCCCCC(C)(C)c1cc(O)c([C@H]2C=C(CC)CC[C@H]2C(C)C)c(O)c1] | Com- pound 50 |
| 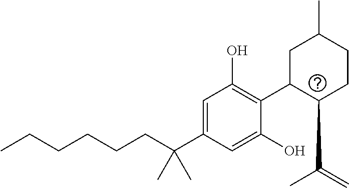 [SMILES: C=C(C)[C@@H]1CCC(C)CC1c1c(O)cc(C(C)(C)CCCCCC)cc1O] | Com- pound 51 |
| 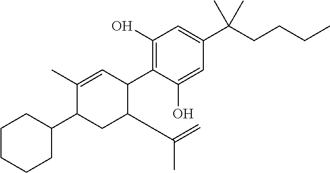 [SMILES: C=C(C)C1CC(C2CCCCC2)C(C)=CC1c1c(O)cc(C(C)(C)CCCC)cc1O] | Com- pound 52 |
| 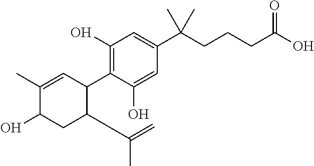 [SMILES: C=C(C)C1CC(O)C(C)=CC1c1c(O)cc(C(C)(C)CCCC(=O)O)cc1O] | Com- pound 53 |

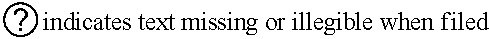

TABLE 2

| | |
|---|---|
| 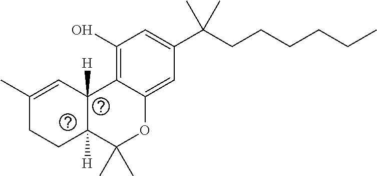 [SMILES: CCCCCCC(C)(C)c1cc(O)c2c(c1)OC(C)(C)[C@H]1CCC(C)=C[C@H]21] | Compound 54 |
| 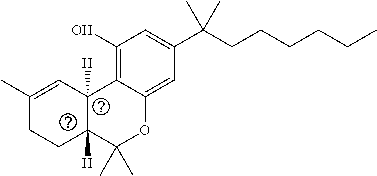 [SMILES: CCCCCCC(C)(C)c1cc(O)c2c(c1)OC(C)(C)[C@@H]1CCC(C)=C[C@@H]21] | Compound 55 |
| 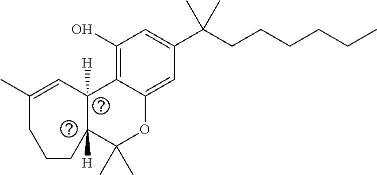 [SMILES: CCCCCCC(C)(C)c1cc(O)c2c(c1)OC(C)(C)[C@@H]1CCCC(C)=C[C@@H]21] | Compound 56 |
| 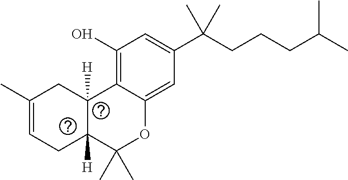 [SMILES: CC1=CC[C@@H]2[C@@H](C1)c1c(O)cc(C(C)(C)CCCC(C)C)cc1OC2(C)C] | Compound 57 |
| 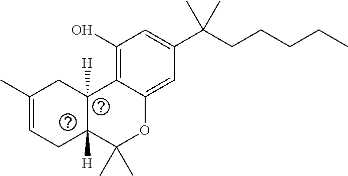 [SMILES: CCCCCC(C)(C)c1cc(O)c2c(c1)OC(C)(C)[C@@H]1CC=C(C)C[C@@H]21] | Compound 58 |
| 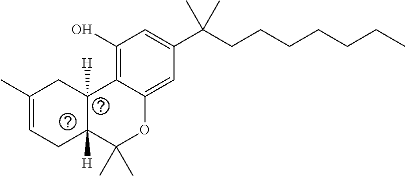 [SMILES: CCCCCCCC(C)(C)c1cc(O)c2c(c1)OC(C)(C)[C@@H]1CC=C(C)C[C@@H]21] | Compound 59 |
| 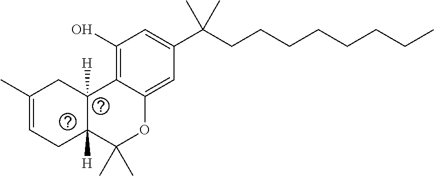 [SMILES: CCCCCCCCC(C)(C)c1cc(O)c2c(c1)OC(C)(C)[C@@H]1CC=C(C)C[C@@H]21] | Compound 60 |
| 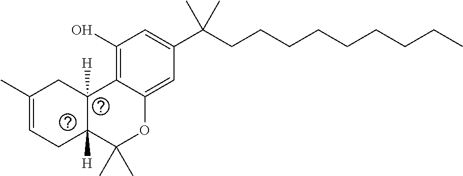 [SMILES: CCCCCCCCCC(C)(C)c1cc(O)c2c(c1)OC(C)(C)[C@@H]1CC=C(C)C[C@@H]21] | Compound 61 |
| 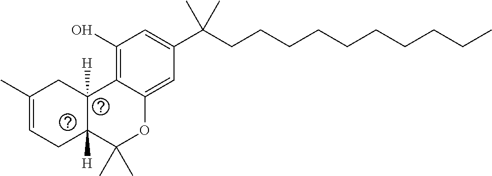 [SMILES: CCCCCCCCCCC(C)(C)c1cc(O)c2c(c1)OC(C)(C)[C@@H]1CC=C(C)C[C@@H]21] | Compound 62 |
| 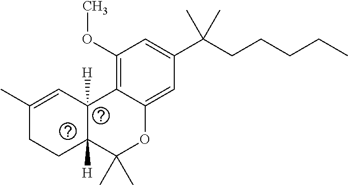 [SMILES: CCCCCC(C)(C)c1cc(OC)c2c(c1)OC(C)(C)[C@@H]1CCC(C)=C[C@@H]21] | Compound 63 |
| 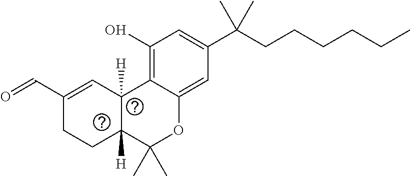 [SMILES: CCCCCCC(C)(C)c1cc(O)c2c(c1)OC(C)(C)[C@@H]1CCC(C=O)=C[C@@H]21] | Compound 64 |
| 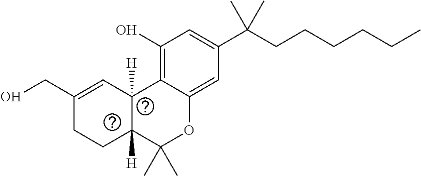 [SMILES: CCCCCCC(C)(C)c1cc(O)c2c(c1)OC(C)(C)[C@@H]1CCC(CO)=C[C@@H]21] | Compound 65 |
| 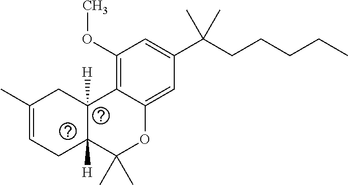 [SMILES: CCCCCC(C)(C)c1cc(OC)c2c(c1)OC(C)(C)[C@@H]1CC=C(C)C[C@@H]21] | Compound 66 |
| 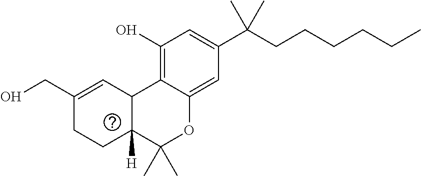 [SMILES: CCCCCCC(C)(C)c1cc(O)c2c(c1)OC(C)(C)[C@@H]1CCC(CO)=CC21] | Compound 67 |
| 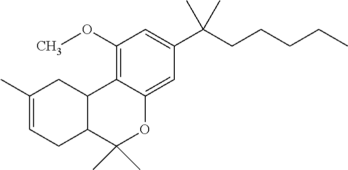 [SMILES: CCCCCC(C)(C)c1cc(OC)c2c(c1)OC(C)(C)C1CC=C(C)CC21] | Compound 68 |
| 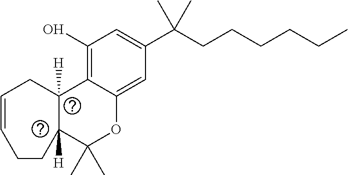 [SMILES: CCCCCCC(C)(C)c1cc(O)c2c(c1)OC(C)(C)[C@@H]1CCC=CC[C@@H]21] | Compound 69 |
| 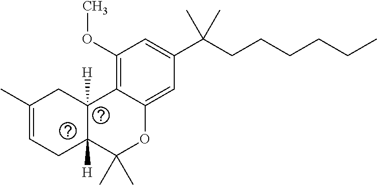 [SMILES: CCCCCCC(C)(C)c1cc(OC)c2c(c1)OC(C)(C)[C@@H]1CC=C(C)C[C@@H]21] | Compound 70 |
| 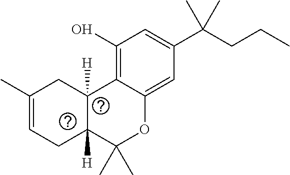 [SMILES: CCCC(C)(C)c1cc(O)c2c(c1)OC(C)(C)[C@@H]1CC=C(C)C[C@@H]21] | Compound 71 |
| 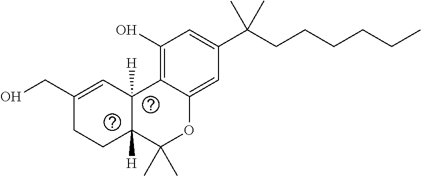 [SMILES: CCCCCCC(C)(C)c1cc(O)c2c(c1)OC(C)(C)[C@@H]1CCC(CO)=C[C@@H]21] | Compound 72 |
| 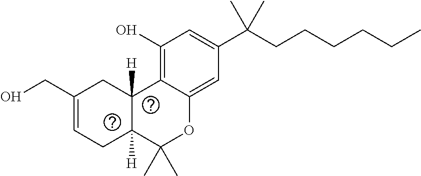 [SMILES: CCCCCCC(C)(C)c1cc(O)c2c(c1)OC(C)(C)[C@H]1CC=C(CO)C[C@H]21] | Compound 73 |
| 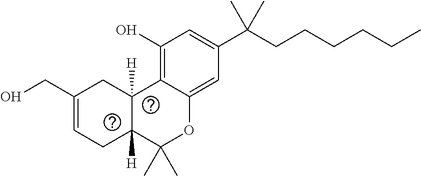 [SMILES: CCCCCCC(C)(C)c1cc(O)c2c(c1)OC(C)(C)[C@@H]1CC=C(CO)C[C@@H]21] | Compound 74 |
| 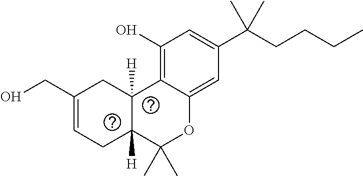 [SMILES: CCCCC(C)(C)c1cc(O)c2c(c1)OC(C)(C)[C@@H]1CC=C(CO)C[C@@H]21] | Compound 75 |
| 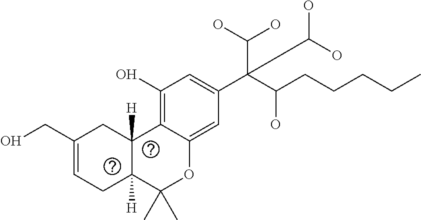 [SMILES: CCCCCC(O)C(c1cc(O)c2c(c1)OC(C)(C)[C@H]1CC=C(CO)C[C@H]21)(C(O)O)C(O)O] | Compound 76 |
| 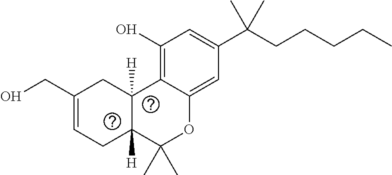 [SMILES: CCCCCC(C)(C)c1cc(O)c2c(c1)OC(C)(C)[C@@H]1CC=C(CO)C[C@@H]21] | Compound 77 |
| 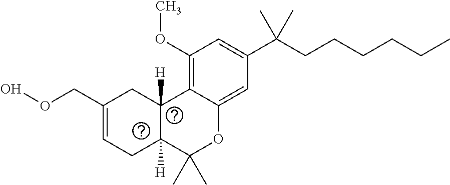 [SMILES: CCCCCCC(C)(C)c1cc(OC)c2c(c1)OC(C)(C)[C@H]1CC=C(COO)C[C@H]21] | Compound 78 |
| 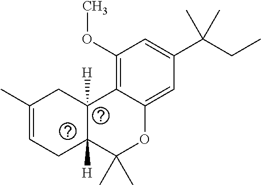 [SMILES: CCC(C)(C)c1cc(OC)c2c(c1)OC(C)(C)[C@@H]1CC=C(C)C[C@@H]21] | Compound 79 |
| 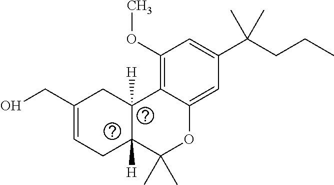 [SMILES: CCCC(C)(C)c1cc(OC)c2c(c1)OC(C)(C)[C@@H]1CC=C(CO)C[C@@H]21] | Compound 80 |
| 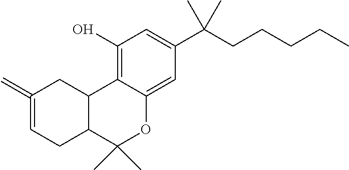 | Compound 81 |
| 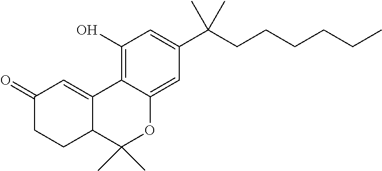 [SMILES: CCCCCCC(C)(C)c1cc(O)c2c(c1)OC(C)(C)C1CCC(=O)C=C21] | Compound 82 |
| 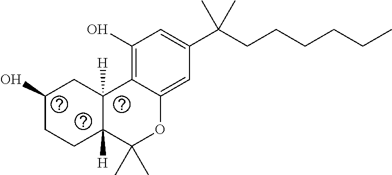 [SMILES: CCCCCCC(C)(C)c1cc(O)c2c(c1)OC(C)(C)[C@@H]1CC[C@@H](O)C[C@@H]21] | Compound 83 |
| 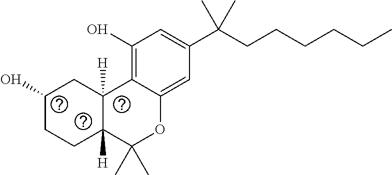 [SMILES: CCCCCCC(C)(C)c1cc(O)c2c(c1)OC(C)(C)[C@@H]1CC[C@H](O)C[C@@H]21] | Compound 84 |
| 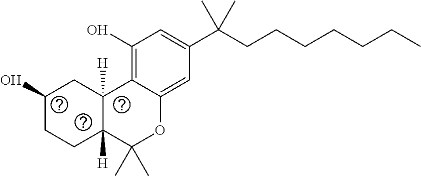 [SMILES: CCCCCCCC(C)(C)c1cc(O)c2c(c1)OC(C)(C)[C@@H]1CC[C@@H](O)C[C@@H]21] | Compound 85 |
| 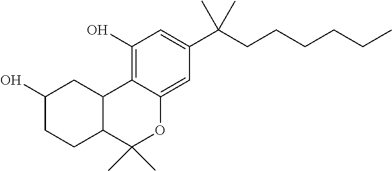 [SMILES: CCCCCCC(C)(C)c1cc(O)c2c(c1)OC(C)(C)C1CCC(O)CC21] | Compound 86 |
| 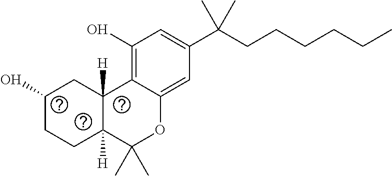 [SMILES: CCCCCCC(C)(C)c1cc(O)c2c(c1)OC(C)(C)[C@H]1CC[C@H](O)C[C@H]21] | Compound 87 |
| 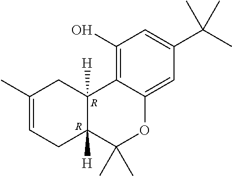 | Compound 88 |
| 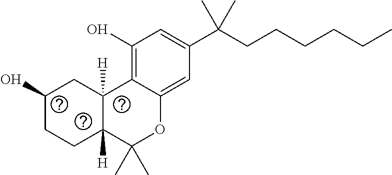 [SMILES: CCCCCCC(C)(C)c1cc(O)c2c(c1)OC(C)(C)[C@@H]1CC[C@@H](O)C[C@@H]21] | Compound 89 |
| 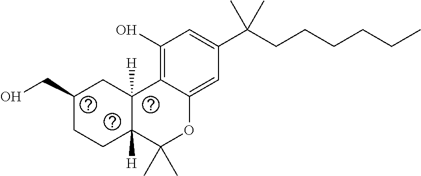 [SMILES: CCCCCCC(C)(C)c1cc(O)c2c(c1)OC(C)(C)[C@@H]1CC[C@@H](CO)C[C@@H]21] | Compound 90 |
| 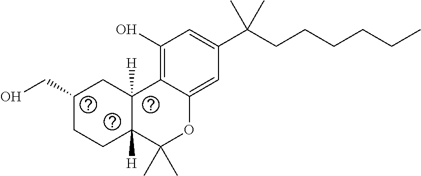 [SMILES: CCCCCCC(C)(C)c1cc(O)c2c(c1)OC(C)(C)[C@@H]1CC[C@H](CO)C[C@@H]21] | Compound 91 |
| 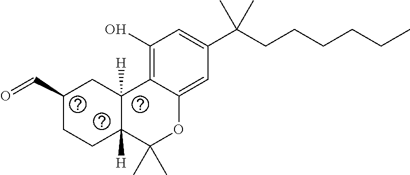 [SMILES: CCCCCCC(C)(C)c1cc(O)c2c(c1)OC(C)(C)[C@@H]1CC[C@@H](C=O)C[C@@H]21] | Compound 92 |
| 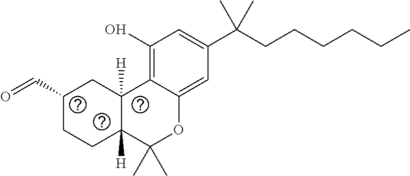 [SMILES: CCCCCCC(C)(C)c1cc(O)c2c(c1)OC(C)(C)[C@@H]1CC[C@H](C=O)C[C@@H]21] | Compound 93 |
| 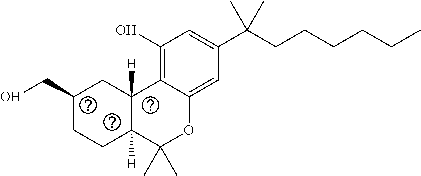 [SMILES: CCCCCCC(C)(C)c1cc(O)c2c(c1)OC(C)(C)[C@H]1CC[C@@H](CO)C[C@H]21] | Compound 94 |
| 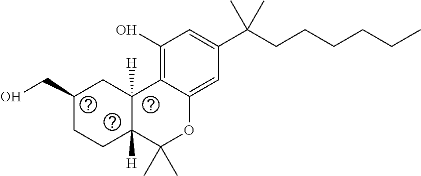 [SMILES: CCCCCCC(C)(C)c1cc(O)c2c(c1)OC(C)(C)[C@@H]1CC[C@@H](CO)C[C@@H]21] | Compound 95 |
| 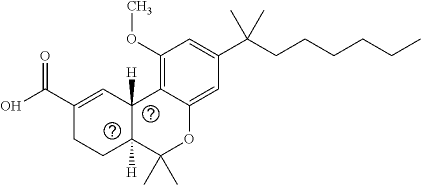 [SMILES: CCCCCCC(C)(C)c1cc(OC)c2c(c1)OC(C)(C)[C@H]1CCC(C(=O)O)=C[C@H]21] | Compound 96 |
| 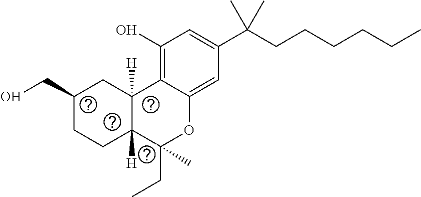 [SMILES: CCCCCCC(C)(C)c1cc(O)c2c(c1)O[C@@](C)(CC)[C@@H]1CC[C@@H](CO)C[C@@H]21] | Compound 97 |
| 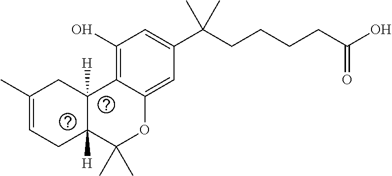 [SMILES: CC1=CC[C@@H]2[C@@H](C1)c1c(O)cc(C(C)(C)CCCCC(=O)O)cc1OC2(C)C] | Compound 98 |
| 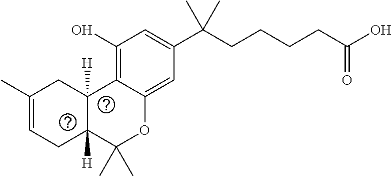 [SMILES: CC1=CC[C@@H]2[C@@H](C1)c1c(O)cc(C(C)(C)CCCCC(=O)O)cc1OC2(C)C] | Compound 99 |
| 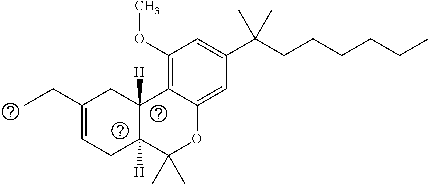 [SMILES: CCCCCCC(C)(C)c1cc(OC)c2c(c1)OC(C)(C)[C@H]1CC=C(CC)C[C@H]21] | Compound 100 |
| 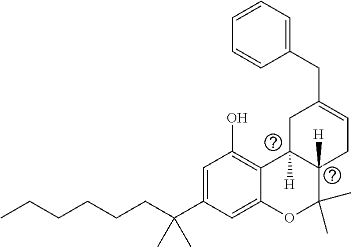 | Compound 101 |
| 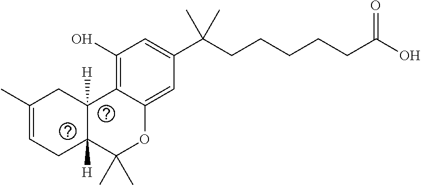 [SMILES: CC1=CC[C@@H]2[C@@H](C1)c1c(O)cc(C(C)(C)CCCCCC(=O)O)cc1OC2(C)C] | Compound 102 |
| 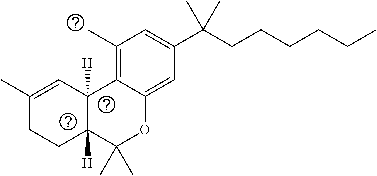 [SMILES: CCCCCCC(C)(C)c1cc(C)c2c(c1)OC(C)(C)[C@@H]1CCC(C)=C[C@@H]21] | Compound 103 |
| 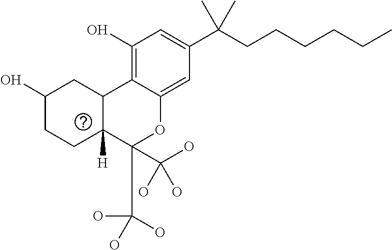 [SMILES: CCCCCCC(C)(C)c1cc(O)c2c(c1)OC(C(O)(O)O)(C(O)(O)O)[C@@H]1CCC(O)CC21] | Compound 104 |
| 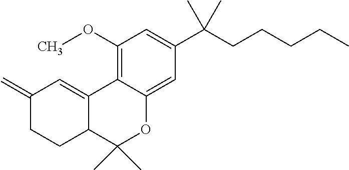 [SMILES: C=C1C=C2c3c(OC)cc(C(C)(C)CCCCC)cc3OC(C)(C)C2CC1] | Compound 105 |
| 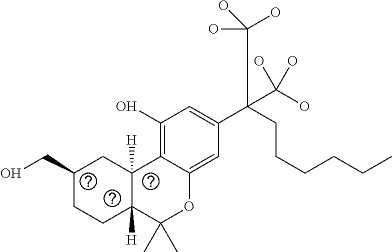 [SMILES: CCCCCCC(c1cc(O)c2c(c1)OC(C)(C)[C@@H]1CC[C@@H](CO)C[C@@H]21)(C(O)(O)O)C(O)(O)O] | Compound 106 |
| 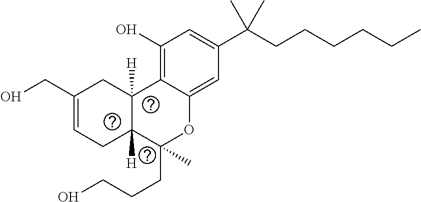 [SMILES: CCCCCCC(C)(C)c1cc(O)c2c(c1)O[C@@](C)(CCCO)[C@@H]1CC=C(CO)C[C@@H]21] | Compound 107 |
| 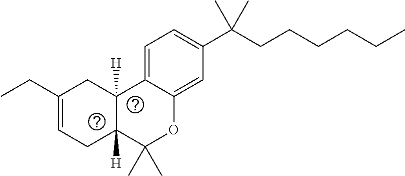 [SMILES: CCCCCCC(C)(C)c1ccc2c(c1)OC(C)(C)[C@@H]1CC=C(CC)C[C@@H]21] | Compound 108 |
| 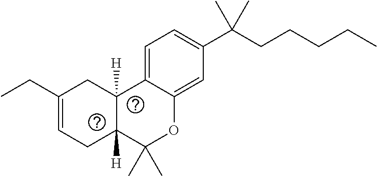 [SMILES: CCCCCC(C)(C)c1ccc2c(c1)OC(C)(C)[C@@H]1CC=C(CC)C[C@@H]21] | Compound 109 |
| 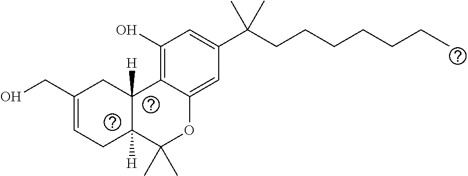 [SMILES: CCCCCCCC(C)(C)c1cc(O)c2c(c1)OC(C)(C)[C@H]1CC=C(CO)C[C@H]21] | Compound 110 |
| 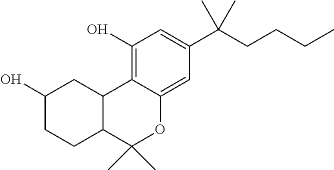 [SMILES: CCCCC(C)(C)c1cc(O)c2c(c1)OC(C)(C)C1CCC(O)CC21] | Compound 111 |
| 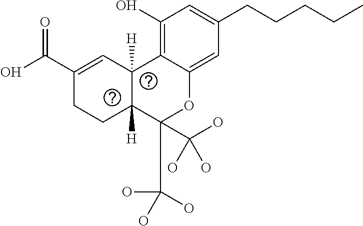 [SMILES: CCCCCc1cc(O)c2c(c1)OC(C(O)(O)O)(C(O)(O)O)[C@@H]1CCC(C(=O)O)=C[C@@H]21] | Compound 112 |
| 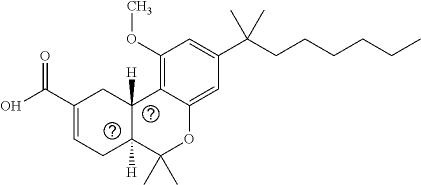 [SMILES: CCCCCCC(C)(C)c1cc(OC)c2c(c1)OC(C)(C)[C@H]1CC=C(C(=O)O)C[C@H]21] | Compound 113 |
| 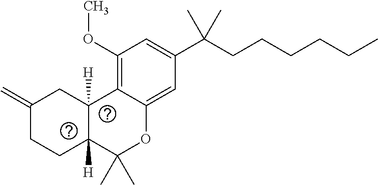 [SMILES: C=C1CC[C@@H]2[C@@H](C1)c1c(OC)cc(C(C)(C)CCCCCC)cc1OC2(C)C] | Compound 114 |
| 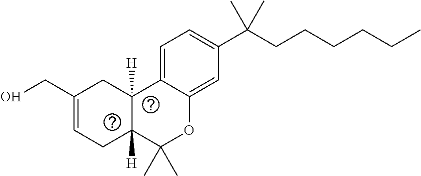 [SMILES: CCCCCCC(C)(C)c1ccc2c(c1)OC(C)(C)[C@@H]1CC=C(CO)C[C@@H]21] | Compound 115 |
| 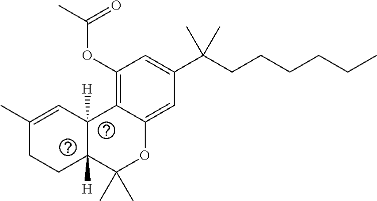 [SMILES: CCCCCCC(C)(C)c1cc(OC(C)=O)c2c(c1)OC(C)(C)[C@@H]1CCC(C)=C[C@@H]21] | Compound 116 |
| 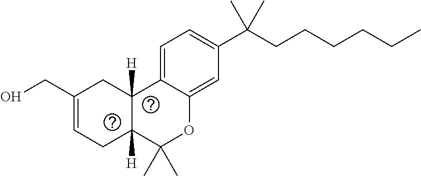 [SMILES: CCCCCCC(C)(C)c1ccc2c(c1)OC(C)(C)[C@@H]1CC=C(CO)C[C@H]21] | Compound 117 |
| 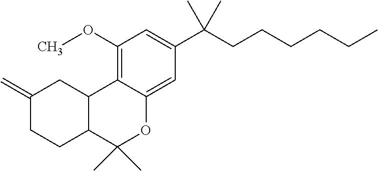 [SMILES: C=C1CCC2C(C1)c1c(OC)cc(C(C)(C)CCCCCC)cc1OC2(C)C] | Compound 118 |
| 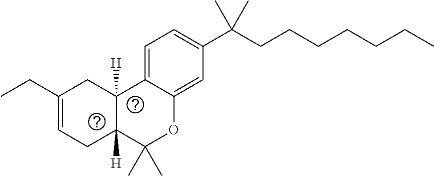 [SMILES: CCCCCCCC(C)(C)c1ccc2c(c1)OC(C)(C)[C@@H]1CC=C(CC)C[C@@H]21] | Compound 119 |
| 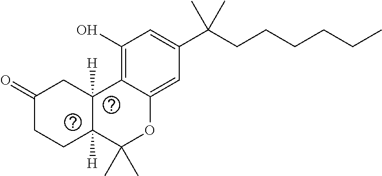 [SMILES: CCCCCCC(C)(C)c1cc(O)c2c(c1)OC(C)(C)[C@H]1CCC(=O)C[C@@H]21] | Compound 120 |
| 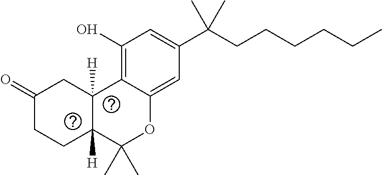 [SMILES: CCCCCCC(C)(C)c1cc(O)c2c(c1)OC(C)(C)[C@@H]1CCC(=O)C[C@@H]21] | Compound 121 |
| 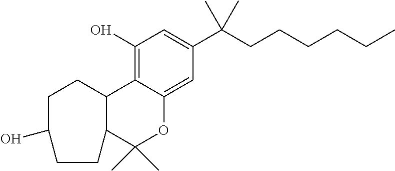 [SMILES: CCCCCCC(C)(C)c1cc(O)c2c(c1)OC(C)(C)C1CCC(O)CCC21] | Compound 122 |
| 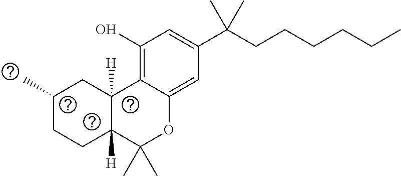 [SMILES: CCCCCCC(C)(C)c1cc(O)c2c(c1)OC(C)(C)[C@@H]1CC[C@H](C)C[C@@H]21] | Compound 123 |
| 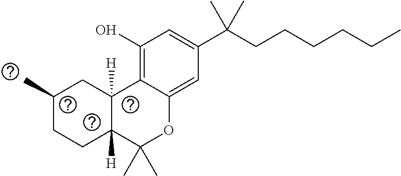 [SMILES: CCCCCCC(C)(C)c1cc(O)c2c(c1)OC(C)(C)[C@@H]1CC[C@@H](C)C[C@@H]21] | Compound 124 |
| 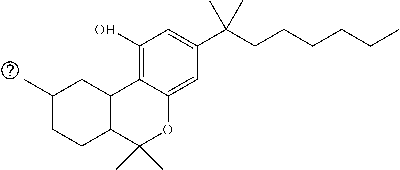 [SMILES: CCCCCCC(C)(C)c1cc(O)c2c(c1)OC(C)(C)C1CCC(C)CC21] | Compound 125 |

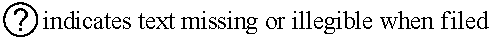

### Pharmaceutical Compositions

Ajulemic acid prepared by any of the methods described herein (e.g., ajulemic acid with reduced levels of homologous alkyl-chain impurities) may be formulated as a pharmaceutical composition for the treatment of disease. As described above, the pharmaceutical compositions of the invention additionally include a pharmaceutically acceptable excipient, which, as used herein, includes any and all solvents, diluents, or other liquid vehicle, dispersion or suspension aids, surface active agents, isotonic agents, thickening or emulsifying agents, preservatives, solid binders, and lubricants, as suited to the particular dosage form desired. Remington's Pharmaceutical Sciences, Sixteenth Edition, E. W. Martin (Mack Publishing Co., Easton, Pa., 1980) discloses various excipients used in formulating pharmaceutical compositions and known techniques for the preparation thereof. Except insofar as any conventional excipient medium is incompatible with the compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable excipients include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; natural and synthetic phospholipids, such as soybean and egg yolk phosphatides, lecithin, hydrogenated soy lecithin, dimyristoyl lecithin, dipalmitoyl lecithin, distearoyl lecithin, dioleoyl lecithin, hydroxylated lecithin, lysophosphatidylcholine, cardiolipin, sphingomyelin, phosphatidylcholine, phosphatidyl ethanolamine, diastearoyl phosphatidylethanolamine (DSPE) and its pegylated esters, such as DSPE-PEG750 and, DSPE-PEG2000, phosphatidic acid, phosphatidyl glycerol and phosphatidyl serine. Commercial grades of lecithin which are preferred include those which are available under the trade name Phosal® or Phospholipon® and include Phosal 53 MCT, Phosal 50 PG, Phosal 75 SA, Phospholipon 90H, Phospholipon 90G and Phospholipon 90 NG; soy-phosphatidylcholine (SoyPC) and DSPE-PEG2000 are particularly preferred; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium laury sultate ana magnesium stearate, as well as coloring agents, releasing agents, coating agents, sweetening, flavoring and perfuming agents, preservatives and antioxidants can also be present in the composition, according to the judgment of the formulator.

The above-described composition, in any of the forms described above, can be used for treating fibrotic disease, inflammatory disease, or any other disease or condition described herein. An effective amount refers to the amount of an active compound/agent that is required to confer a therapeutic effect on a treated subject. Effective doses will vary, as recognized by those skilled in the art, depending on the types of diseases treated, route of administration, excipient usage, and the possibility of co-usage with other therapeutic treatment.

A pharmaceutical composition of this invention can be administered parenterally, orally, nasally, rectally, topically, buccally, by ophthalmic administration, or by inhalation. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent. Such solutions include, but are not limited to, 1,3-butanediol, mannitol, water, Ringer's solution, and isotonic sodium chloride solution. In addition, fixed oils are conventionally employed as a solvent or suspending medium (e.g., synthetic mono- or diglycerides). Fatty acids, such as, but not limited to, oleic acid and its glyceride derivatives, are useful in the preparation of injectables, as are natural pharmaceutically acceptable oils, such as, but not limited to, olive oil or castor oil, or polyoxyethylated versions thereof. These oil solutions or suspensions also can contain a long chain alcohol diluent or dispersant such as, but not limited to, carboxymethyl cellulose, or similar dispersing agents. Other commonly used surfactants, such as, but not limited to, Tweens or Spans or other similar emulsifying agents or bioavailability enhancers, which are commonly used in the manufacture of pharmaceutically acceptable solid, liquid, or other dosage forms also can be used for the purpose of formulation.

A composition for oral administration can be any orally acceptable dosage form including capsules, tablets (e.g. a pressed tablet), emulsions and aqueous suspensions, dispersions, and solutions. In the case of tablets, commonly used excipients include, but are not limited to, lactose and corn starch. Lubricating agents, such as, but not limited to, magnesium stearate, also are typically added. For oral administration in a capsule form, useful diluents include, but are not limited to, lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

Pharmaceutical compositions for topical administration according to the described invention can be formulated as solutions, ointments, creams, suspensions, lotions, powders, pastes, gels, sprays, aerosols, or oils. Alternatively, topical formulations can be in the form of patches or dressings impregnated with active ingredient(s), which can optionally include one or more excipients or diluents. In some preferred embodiments, the topical formulations include a material that would enhance absorption or penetration of the active agent(s) through the skin or other affected areas.

A topical composition contains a safe and effective amount of a dermatologically acceptable excipient suitable for application to the skin. A “cosmetically acceptable” or “dermatologically-acceptable” composition or component refers a composition or component that is suitable for use in contact with human skin without undue toxicity, incompatibility, instability, or allergic response. The excipient enables an active agent and optional component to be delivered to the skin at an appropriate concentration(s). The excipient thus can act as a diluent, dispersant, solvent, or the like to ensure that the active materials are applied to and distributed evenly over the selected target at an appropriate concentration. The excipient can be solid, semi-solid, or liquid. The excipient can be in the form of a lotion, a cream, or a gel, in particular one that has a sufficient thickness or yield point to prevent the active materials from sedimenting. The excipient can be inert or possess dermatological benefits. It also should be physically and chemically compatible with the active components described herein, and should not unduly impair stability, efficacy, or other use benefits associated with the composition.

### Pharmaceutical Dosage Forms

Various dosage forms of ajulemic acid (e.g., ajulemic acid produced by any of the methods described herein) can be used for preventing and/or treating a condition (e.g., an inflammatory disease or a fibrotic disease). In some embodiments, the dosage form is an oral dosage form such as a pressed tablet, hard or soft gel capsule, enteric coated tablet, osmotic release capsule, or unique combination of excipients.

In further embodiments, the dosage form includes an additional agent or is provided together with a second dosage form, which includes the additional agent. Exemplary additional agents include an analgesic agent such as an NSAID or opiate, an anti-inflammatory agent or a natural agent such as a triglyceride containing unsaturated fatty acid, or isolated pure fatty acids such as eicosapentaenoic acid (EPA), dihomogamma linolenic acid (DGLA), docosahexaenoic acid (DHA) and others. In additional embodiments, the dosage form includes a capsule wherein the capsule contains a mixture of materials to provide a desired sustained release formulation.

The dosage forms can include a tablet coated with a semipermeable coating. In certain embodiments, the tablet includes two layers, a layer containing ajulemic acid (e.g. ultrapure ajulemic acid) and a second layer referred to as a “push” layer. The semi-permeable coating is used to allow a fluid (e.g., water) to enter the tablet and erode a layer or layers. In certain embodiments, this sustained release dosage form further includes a laser hole drilled in the center of the coated tablet. The ajulemic acid containing layer may include ajulemic acid, a disintegrant, a viscosity enhancing agent, a binding agent, and an osmotic agent. The push layer includes a disintegrant, a binding agent, an osmotic agent, and a viscosity enhancing agent.

The present compositions may be formulated for sustained release (e.g., over a 2 hour period, over a 6 hour period, over a 12 hour period, over a 24 hour period, or over a 48 hour period).

In further embodiments, the dosage form includes a tablet including a biocompatible matrix and ajulemic acid. The sustained release dosage form may also include a hard-shell capsule containing bio-polymer microspheres that contains the therapeutically active agent. The biocompatible matrix and bio-polymer microspheres each contain pores for drug release and delivery. These pores are formed by mixing the biocompatible matrix of bio-polymer microsphere with a pore forming agent. Each biocompatible matrix or bio-polymer microsphere is made up of a biocompatiDle polymer or mixture of biocompatible polymers. The matrix and microspheres can be formed by dissolving the biocompatible polymer and active agent (compound described herein) in a solvent and adding a pore-forming agent (e.g., a volatile salt). Evaporation of the solvent and pore forming agent provides a matrix or microsphere containing the active compound. In additional embodiments, the sustained release dosage form includes a tablet, wherein the tablet contains ajulemic acid and one or more polymers and wherein the tablet can be prepared by compressing the ajulemic acid and one or more polymers. In some embodiments, the one or more polymers may include a hygroscopic polymer formulated with ajulemic acid. Upon exposure to moisture, the tablet dissolves and swells. This swelling allows the sustained release dosage form to remain in the upper GI tract. The swelling rate of the polymer mixture can be varied using different grades of polyethylene oxide.

In other embodiments, the sustained release dosage form includes a capsule further including particle cores coated with a suspension of active agent and a binding agent which is subsequently coated with a polymer. The polymer may be a rate-controlling polymer. In general, the delivery rate of the rate-controlling polymer is determined by the rate at which the active agent is dissolved.

In some embodiments, one or more of the therapeutic agents that can be used for preventing and/or treating fibrotic disease or inflammatory disease may be formulated with a pharmaceutically acceptable carrier, vehicle or adjuvant. The term “pharmaceutically acceptable carrier, vehicle, or adjuvant” refers to a carrier, vehicle or adjuvant that may be administered to a subject, together with the present compounds, and which does not destroy the pharmacological activity thereof and is nontoxic when administered in doses sufficient to deliver a therapeutic amount of the compound.

Pharmaceutically acceptable carriers, adjuvants and vehicles that may be used in the dosage forms of this invention include, but are not limited to, ion exchangers, alumina, aluminum stearate, lecithin, self-emulsifying drug delivery systems (SEDDS) such as d-E-tocopherol polyethylene-glycol 1000 succinate; surfactants used in pharmaceutical dosage forms such as Tweens or other similar polymeric delivery matrices; serum proteins such as human serum albumin; buffer substances such as phosphates, glycine, sorbic acid, potassium sorbate, partial glyceride mixtures of saturated vegetable fatty acids, water, salts; or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxmethylcellulose, polyacrylates, waxes, polyethylene-polyoxypropylene-block polymers, polyethylene glycol and wool fat.

Cyclodextrins such as alpha, beta and .gamma.-cyclodextrin, or chemically modified derivatives such as hydroxyalkylcyclodextrins, including 2- and 3-hydroxypropyl-beta cyclodextrins, or other solubilized derivatives may also be advantageously used to enhance delivery of compounds of the formulae described herein that can be used in the methods of the invention for preventing and/or treating fibrotic conditions. In certain embodiments, unit dosage formulations are compounded for immediate release, though unit dosage formulations compounded for delayed or prolonged release of one or both agents are also disclosed.

In some embodiments, ajulemic acid may be formulated in a single unit dose such that the agents are released from the dosage at different times.

In another embodiment, for example, where one or more of the therapeutic agents is administered once or twice per day, the agent is formulated to provide extended release. For example, the agent is formulated with an enteric coating. In an alternative embodiment, the agent is formulated using a biphasic controlled release delivery system, thereby providing prolonged gastric residence. For example, in some embodiments, the delivery system includes (1) an inner solid particulate phase formed of substantially uniform granules containing a pharmaceutical having a high water solubility, and one or more hydrophilic polymers, one or more hydrophobic polymers and/or one or more hydrophobic materials such as one or more waxes, fatty alcohols and/or fatty acid esters, and (2) an outer solid continuous phase in which the above granules of inner solid particulate phase are embedded and dispersed throughout, the outer solid continuous phase including one or more hydrophobic polymers, one or more hydrophobic polymers and/or one or more hydrophobic materials such as one or more waxes, fatty alcohols and/or fatty acid esters, which may be compressed into tablets or filled into capsules. In some embodiments, the agent is incorporated into polymeric matrices comprised of hydrophilic polymers that swell upon imbibition of water to a size that is large enough to promote retention of the dosage form in the stomach during the fed mode.

The ajulemic acid in the formulation may be formulated as a combination of fast-acting and controlled release forms. For example, the ajulemic acid is formulated with a single release property. For example, it is not present in a modified release form, e.g., a controlled release form.

The present compositions may be taken just prior to or with each of three meals, each of two major meals, or one meal. In other embodiments, a composition disclosed herein can be administered one or more times daily (e.g., once daily, twice daily, or three times daily) and need not be administered just before or with a meal.

The present compounds or compositions may be administered orally, for example as a component in a dosage form. The dosage forms may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form.

The dosage forms of this invention may be orally administered in any orally acceptable dosage form including, but not limited to, capsules, tablets, emulsions and aqueous suspensions, dispersions and solutions. In the case of tablets for oral use, carriers that are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

Non-limiting examples of capsules include but are not limited to gelatin capsules, HPMC, hard shell, soft shell, or any other suitable capsule for holding a sustained release mixture. The solvents used in the above sustained release dosage forms include, but are not limited to ethyl acetate, triacetin, dimethyl sulfoxide (DIV1S0), propylene carbonate, N-methylpyrrolidone (NMP), ethyl alcohol, benzyl alcohol, glycofurol, alpha-tocopherol, Miglyol 810, isopropyl alcohol, diethyl phthalate, polyethylene glycol 400 (PEG 400), triethyl citrate, and benzyl benzoate.

The viscosity modifiers that may be used in the above pharmaceutical compositions include, but are not limited to caprylic/capric triglyceride (Migliol 810), isopropyl myristate (IPM), ethyl oleate, triethyl citrate, dimethyl phthalate, benzyl benzoate and various grades of polyethylene oxide. The high viscosity liquid carriers used in the above sustained release dosage forms include, but are not limited to sucrose acetate isobutyrate (SA1B) and cellulose acetate butyrate (CAB) 381-20.

Non-limiting examples of materials that make up preferred semi-permeable layers include, but are not limited to cellulosic polymers such as cellulose acetate, cellulose acylate, cellulose diacylate, cellulose triacylate, cellulose diacetate, cellulose triacetate or any mixtures thereof; ethylene vinyl acetate copolymers, polyethylene, copolymers of ethylene, polyolefins including ethylene oxide copolymers (e.g., Engage®—Dupont Dow Elastomers), polyamides, cellulosic materials, polyurethanes, polyether blocked amides, and copolymers (e.g., PEBAX®, cellulosic acetate butyrate and polyvinyl acetate). Non-limiting examples of disintegrants that may be employed in the above sustained release dosage forms include but are not limited to croscarmellose sodium, crospovidone, sodium alginate or similar excipients.

Non-limiting examples of binding agents that may be employed in the above dosage forms include but are not limited to hydroxyalkylcellulose, a hydroxyalkylalkylcellulose, or a polyvinylpyrrolidone.

Non-limiting examples of osmotic agents that may be employed in the above dosage forms include but are not limited to, sorbitol, mannitol, sodium chloride, or other salts. Non-limiting examples of biocompatible polymers employed in the above sustained release dosage forms include but are not limited to poly(hydroxy acids), polyanhydrides, polyorthoesters, polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polysiloxanes, poly(vinyl alcohols), poly (vinyl acetate), polystyrene, polyurethanes and co-polymers thereof, synthetic celluloses, polyacrylic acids, poly(butyric acid), poly(valeric acid), and poly(lactide-co-caprolactone), ethylene vinyl acetate, copolymers and blends thereof.

Non-limiting examples of hygroscopic polymers that may be employed in the above dosage forms include but are not limited to polyethylene oxide (e.g., Polyox® with MWs from 4,000,000 to 10,000,000), cellulose hydroxymethyl cellulose, hydroxyethyl-cellulose, crosslinked polyacrylic acids and xanthum gum.

Non-limiting examples of rate-controlling polymers the may be employed in the above dosage forms include but are not limited to polymeric acrylate, methacrylate lacquer or mixtures thereof, polymeric acrylate lacquer, methacrylate lacquer, an acrylic resin including a copolymer of acrylic and methacrylic acid esters or an ammonium methacrylate lacquer with a plasticizer.

### Methods of Treatment

In some embodiments of the invention, any of the above-described compositions (e.g., compositions including ajulemic acid prepared according to the methods of the invention), including any of the above-described pharmaceutical compositions, may be administered to a subject (e.g., a mammal, such as a human, cat, dog, horse, cow, or pig) having a disease (e.g., a fibrotic disease or an inflammatory disease) in order to treat, prevent, or ameliorate the disease.

Inflammation

Inflammatory diseases include, for example, systemic lupus erythematosus, AIDs, multiple sclerosis, rheumatoid arthritis, psoriasis, diabetes (e.g., Type 1 diabetes), cancer, asthma, atopic dermatitis, autoimmune thyroid disorders, ulcerative colitis, Crohn's disease, stroke, ischemia, and neurodegenerative diseases, (e.g., Alzheimer's disease and Parkinson's disease), ALS, CTE, chronic inflammatory demyelinating polyneuropathy, Autoimmune inner ear disease, Uveitis, iritis, and peritonitis.

In some embodiments, inflammation can be assayed by measuring the chemotaxis and activation state of inflammatory cells. In some embodiments, inflammation can be measured by examining the production of specific inflammatory mediators such as interleukins, cytokines and eicosanoids mediators.

In some embodiments, in vivo inflammation is measured by swelling and edema of a localized tissue or migration of leukocytes. Inflammation may also be measured by organ function such as in the lung or kidneys and by the production of pro-inflammatory factors. Inflammation may also be assessed by other suitable methods. Other methods known to one skilled in the art may also be suitable methods for the assessment of inflammation and may be used to evaluate or score the response of the subject to treatment with ajulemic acid.

Fibrotic Diseases

Fibrotic diseases include, for example, scleroderma, systemic sclerosis, scleroderma-like disorders, sine scleroderma, liver cirrhosis, interstitial pulmonary fibrosis, idiopathic pulmonary fibrosis, Dupuytren's contracture, keloids, cystic fibrosis, chronic kidney disease, chronic graft rejection, fibrosis of organs such as liver, esophagus, heart, lung, intestines, etc., scarring/wound healing abnormalities, post-operative adhesions, reactive fibrosis, dermatomyositis, polymyositis, ANCA vasculitis, Behcet's disease, anti-phospholipid syndrome, relapsing polychondritis, Familial Mediterranean Fever, giant cell arteritis, Graves ophthalmopathy, discoid lupus, pemphigus, bullous pemphigoid, hydradenitis suppuritiva, sarcoidosis, bronchiolitis obliterans, intersititial lung disease, idiopathic pulmonary fibrosis, primary sclerosing cholangitis, and primary biliary cirrhosis.

Non-limiting examples of fibrosis include liver fibrosis, lung fibrosis (e.g., silicosis, asbestosis, idiopathic pulmonary fibrosis), oral fibrosis, endomyocardial fibrosis, retroperitoneal fibrosis, deltoid fibrosis, kidney fibrosis (including diabetic nephropathy), cystic fibrosis, and glomerulosclerosis. Liver fibrosis, for example, occurs as a part of the wound-healing response to chronic liver injury. Fibrosis can occur as a complication of haemochromatosis, Wilson's disease, alcoholism, schistosomiasis, viral hepatitis, bile duct obstruction, exposure to toxins, and metabolic disorders. Endomyocardial fibrosis is an idiopathic disorder that is characterized by the development of restrictive cardiomyopathy. In endomyocardial fibrosis, the underlying process produces patchy fibrosis of the endocardial surface of the heart, leading to reduced compliance and, ultimately, restrictive physiology as the endomyocardial surface becomes more generally involved. Oral submucous fibrosis is a chronic, debilitating disease of the oral cavity characterized by inflammation and progressive fibrosis of the submucosal tissues (lamina propria and deeper connective tissues). The buccal mucosa is the most commonly involved site, but any part of the oral cavity can be involved, even the pharynx. Retroperitoneal fibrosis is characterized by the development of extensive fibrosis throughout the retroperitoneum, typically centered over the anterior surface of the fourth and fifth lumbar vertebrae.

Scleroderma

Scleroderma is a disease of the connective tissue characterized by fibrosis of the skin and internal organs. Scleroderma has a spectrum of manifestations and a variety of therapeutic implications. It includes localized scleroderma, systemic sclerosis, scleroderma-like disorders, and sine scleroderma. Systemic sclerosis can be diffuse or limited. Limited systemic sclerosis is also called CREST (calcinosis, Raynaud's esophageal dysfunction, sclerodactyly, telangiectasia). Systemic sclerosis includes: scleroderma lung disease, scleroderma renal crisis, cardiac manifestations, muscular weakness including fatigue or limited CREST, gastrointestinal dysmotility and spasm, and abnormalities in the central, peripheral and autonomic nervous system.

The major symptoms or manifestations of scleroderma, and in particular of systemic sclerosis, are inappropriate excessive collagen synthesis and deposition, endothelial dysfunction, vasospasm, collapse and obliteration of vessels by fibrosis. In terms of diagnosis, an important clinical parameter may be skin thickening proximal to the metacarpophalangeal joints. Raynaud's phenomenon may be a component of scleroderma. Raynaud's may be diagnosed by color changes of the skin upon cold exposure. Ischemia and skin thickening may also be symptoms of Raynaud's disease.

A therapeutically effective amount of any of the compositions described herein (e.g. ajulemic acid prepared by any of the methods described herein) may be used to treat or prevent fibrosis. Fibrosis may be assessed by suitable methods known to one of skill in the art.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1. Synthesis of DMHR

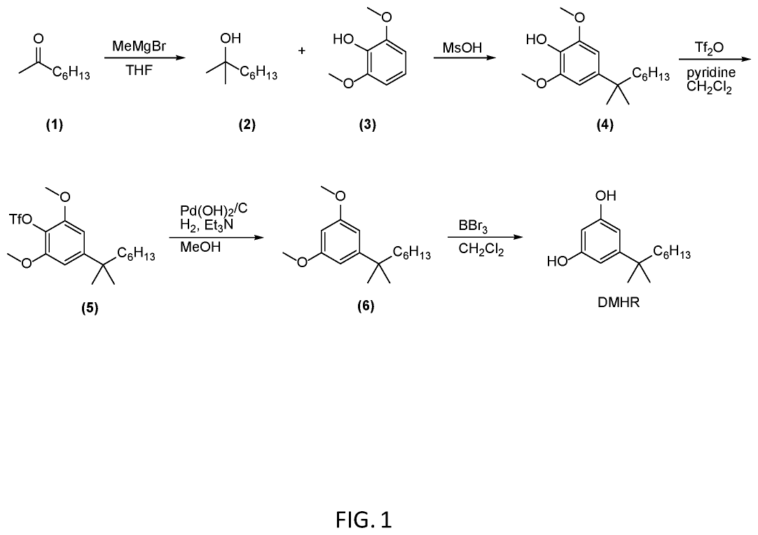

Synthesis of DMHR under original conditions was performed according to the following methods and is further illustrated in the synthetic scheme of FIG. 1.

### Synthesis of Compound (2)

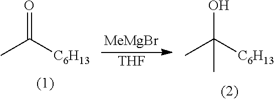

A solution of 2-octanone (20.0 g, 156.0 mmol) in THF (100 mL) was added slowly into a stirred solution of 3M MeMgBr in 2-MeTHF (62.4 mL, 1.2 equiv) in 200 mL THF while cooling the reaction vessel in an ice-water bath. Following the addition, the mixture was stirred at 20-25° C. for 3 hours, at which point the reaction was judged complete by ¹H NMR analysis. The mixture was re-cooled in an ice-water bath and the reaction quenched with aqueous NH₄Cl. The mixture was extracted with EtOAc and the combined extracts were washed with brine and dried over MgSO₄. The dried extracts were concentrated to provide 22.37 g of Compound (2) as an oil.

### Synthesis of Compound (4)

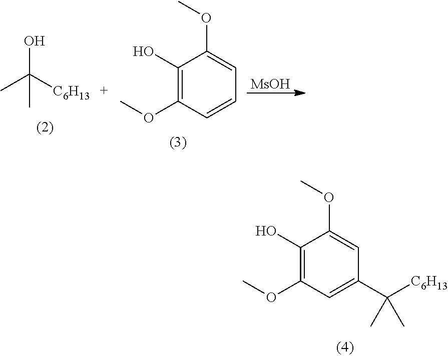

A solution of 2,6-dimethoxyphenol (Compound (3), 5.0 g, 32 mmol) and Compound (2) (5.1 g, 36 mmol, 1.1 equiv) in MsOH (6.3 ml, 9.4 g, 3.0 equiv) was heated to 50° C. for 72 hours, by which time the reaction was judged complete by HPLC analysis. The reaction mixture was cooled in an ice bath and quenched slowly with water. The mixture was extracted with MTBE and the combined extracts were washed with saturated aqueous NaHCO₃ and dried over MgSO₄. The dried extracts were concentrated to afford 8.8 g of Compound (4) as an oil.

### Synthesis of Compound (5)

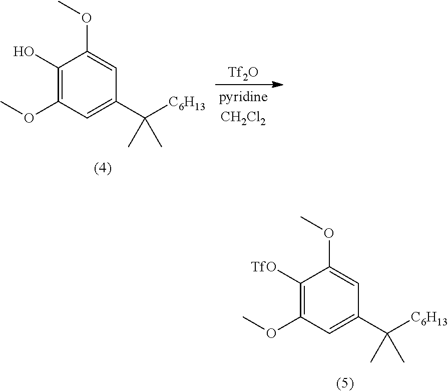

A solution of Compound (4) (1.0 g, 3.6 mmol) and pyridine (0.57 ml, 0.56 g, 2.0 equiv) in CH₂Cl₂ (4 mL) was cooled in an ice bath. Triflic anhydride (0.72 ml, 1.2 g, 1.2 equiv) was added dropwise, after which the reaction mixture was stirred at 20-25° C. for another 30 minutes, by which point the reaction was judged complete by TLC analysis. The reaction mixture was partitioned between water and CH₂Cl₂ and the organic layer washed twice with 1 M HCl and twice with saturated aqueous NaHCO₃, then dried over MgSO₄. Concentration under vacuum provided 1.8 g of Compound (5) as a brown oil.

### Synthesis of Compound (6)

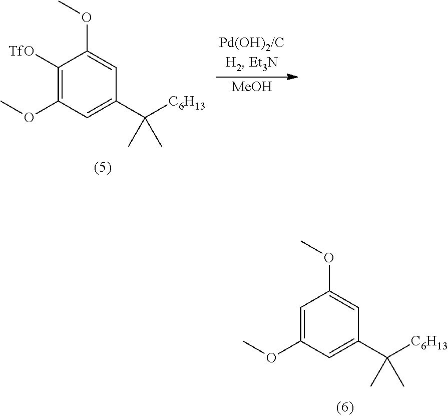

A mixture of Compound (5) (60.0 g, 145 mmol), Et₃N (36.8 g, 2.5 equiv) and 20% Pd(OH)₂/C (wet, 0.11 equiv) in MeOH (600 mL) at 20-25° C. was hydrogenated at 20 psi. After 56 hours, the reaction was judged complete by HPLC analysis. The reaction mixture was filtered through a bed of Celite and the filtrate partitioned between water and CH₂Cl₂. The organic layer was washed twice with 15% (w/w) aqueous NH₄Cl and once with water, then dried over MgSO₄. The solvent was removed under vacuum to produce 37.5 g of Compound (6) as an oil.

### Synthesis of DMHR

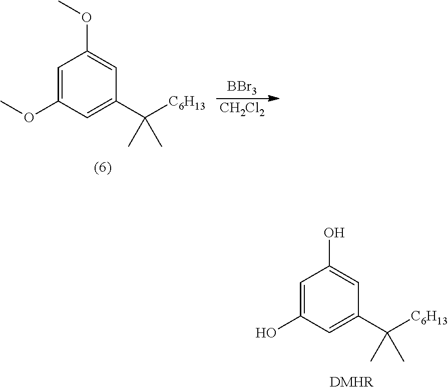

A solution of Compound (6) (2.0 g, 7.6 mmol) in CH2Cl₂ (20 mL) was cooled to −78° C. BBr₃ (1.8 mL, 4.7 g, 2.5 equiv) was added dropwise, after which the reaction mixture was warmed to 20-25° C. and stirred for 5 hours, by which point the reaction was judged complete by HPLC analysis. The reaction mixture was poured into ice water and extracted twice with EtOAc. The combined extracts were washed with water and dried over MgSO₄. The dried extracts were concentrated to a brown oil which was crystallized from a mixture of CH2Cl₂ (4 mL) and heptane (200 mL) to provide 2.0 g of DMHR.

### Example 2. Characterization of DMHR Produced by Original Conditions

DMHR produced by several manufacturers (e.g., according to the protocol of Example 1) was characterized by HPLC to determine the overall purity of the DMHR (% area under the curve, AUC), and to determine the presence of homologous alkyl-chain impurities in the preparations (e.g., one carbon shorter and once carbon longer) (% AUC). Table 3 shows that the commercial preparations of DMHR lack sufficient purity due to the presence of elevated levels of homologous alkyl-chain impurities since such impurities may be carried through to the synthesis of, for example, a therapeutic active such as ajulemic acid.

TABLE 3

| | | One Carbon | One Carbon |
|---|---|---|---|
| | Overall | Shorter | Longer |
| | Purity | Impurity | Impurity |
| Commercial source | (% AUC) | (% AUC) | (% AUC) |
| Chemtarget Technologies | 98.20 | 0.37 | 0.27 |
| Sichuan, China | | | |
| Capot Chemical Company | 96.74 | 0.94 | 0.30 |
| Hangzhou, China | | | |
| Shanghai PI Chemicals | 98.14 | 0.37 | 0.29 |
| Shanghai, China | | | |
| Taian Zhishang Industry | 98.00 | 0.37 | 0.30 |
| Tai'an City, China | | | |
| DMHR specifications to enable | ≥98.0 | ≤0.10 | ≤0.15 |
| production of pharmaceutical | | | |
| grade ajulemic acid | | | |

### DMHR Impurities Quantitation by HPLC

The assay and organic impurities of DMHR were quantitated by gradient elution HPLC using an Agilent Zorbax RX C18, (150 mm×4.6 mm, 5 μm particle size). Mobile phase A was a mixture of water, acetonitrile, and phosphoric acid at a ratio of 60:10:0.1 (v/v/v) respectively. Mobile phase B was a mixture of water, acetonitrile, and phosphoric acid at a ratio of 5:95:0.1 (v/v/v) respectively. The flow rate was set to 1.0 mL/min. The gradient details are outline below in Table 4. The detection wavelength was set to 230 nm. Impurities were calculating by determining the percent area of each impurity as compared to the total chromatographic area.

TABLE 4

| Time (min) | % Mobile Phase A | % Mobile Phase B |
|---|---|---|
| 0.0 | 100 | 0 |
| 3.0 | 100 | 0 |
| 30.0 | 0 | 100 |
| 40.0 | 0 | 100 |
| 42.0 | 100 | 0 |
| 52.0 | 100 | 0 |

### Example 3. Characterization of 2-Octanone Starting Material

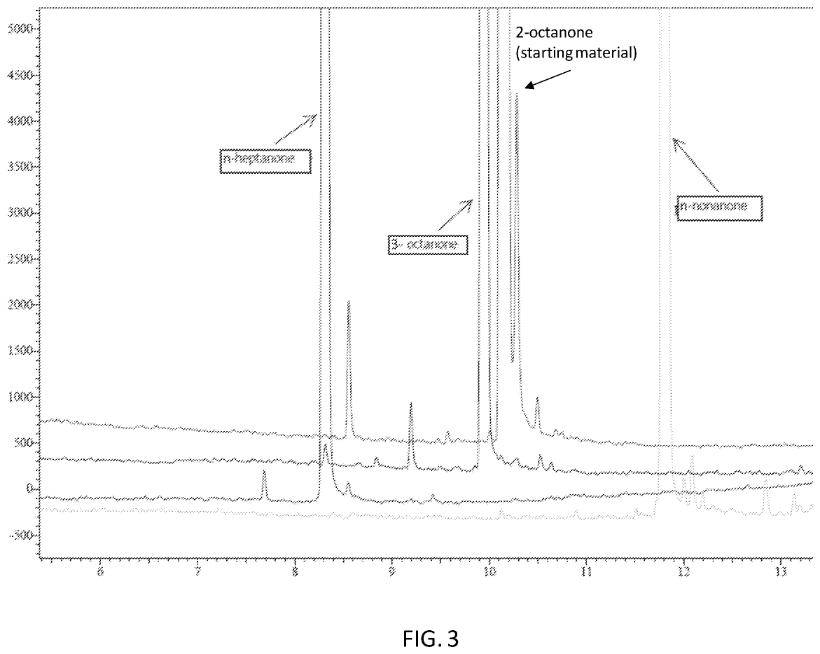

It has been assumed by those skilled in the art that control of homologous alkyl-chain impurities in DMHR is related to the quality of 2-octanone, one of the starting materials in the synthesis of DMHR. High purity 2-octanone (Changzhou Xiaqing Chemical Co., Ltd.) was analyzed by gas chromatography to identify whether significant homologous alkyl-chain impurities in this starting material might be the source of the resulting homologous alkyl-chain impurities observed in the production of DMHR. FIG. 3 shows that no significant amount of several possible alkyl-chain impurities, corresponding to n-heptanone, 3-octanone, or n-nonanone, were observed by gas chromatography analysis of the high purity 2-octanone. Since the use of this high purity 2-octanone still produces homologous alkyl-chain impurities in the resulting DMHR, they are not a result of homologous alkyl-chain impurities in 2-octanone, but rather, are formed as a result of the synthesis.

### 2-Octanone Purity by Gas Chromatography

The purity of 2-octanone was determined using an Shimadzu Gas Chromatograph (GC) configured with a Restek Rtx-1701 capillary column, 30 m (L)×0.25 mm (ID)×0.25 μm (df). A 1.0 μL injection was performed on a 220° C. inlet with a 20:1 split flow. The oven gradient that the column was subjected to during separation is defined below in Table 5. Nitrogen was used as the carrier gas with a constant flow rate of 30 cm/second. Detection was performed using a Flame Ionization Detector (FID) operating at 280° C.

TABLE 5

| Ramp (° C./min) | Hold Time (min) | Final Temperature (° C.) |
|---|---|---|
| N/A | 3 | 45 |
| 10 | 0 | 150 |
| 20 | 0 | 260 |

### Example 4. Characterization of Homologous Alkyl-Chain Impurities in DMHR

DMHR produced according the methods of Example 1, and synthetic intermediates in the production of DMHR, were characterized to determine the identity of several homologous alkyl-chain impurities present in the preparations. Following the coupling of 2-methyloctan-2-ol to 1,3-dimethoxy-2-hydroxybenzene to produce Compound (4) (e.g., Step 2 in the synthesis of DMHR), two impurities having a structure according to Formula (I) were identified by chromatography and characterized by ¹H NMR and mass spectrometry, wherein Formula (I) is

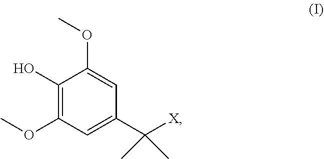

where X is a linear or branched C1-C10 alkyl other than n-C₆H₁₃,

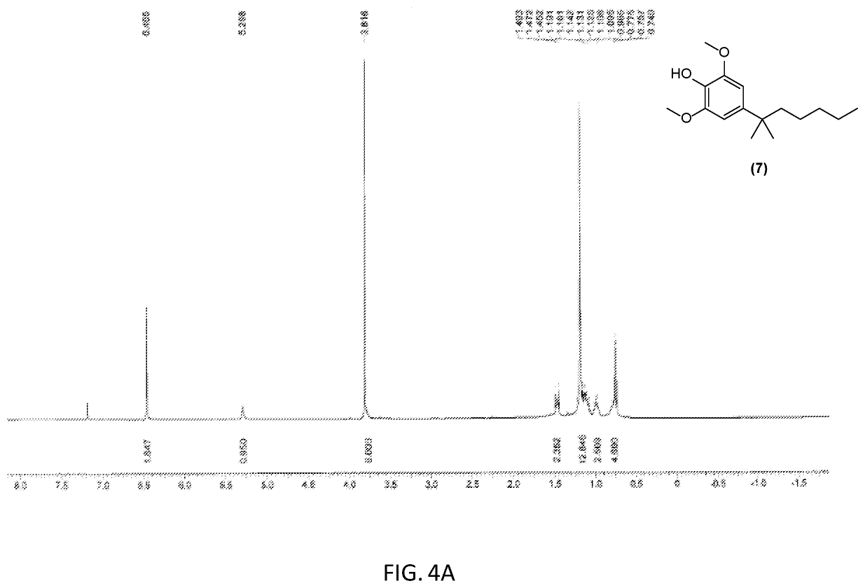

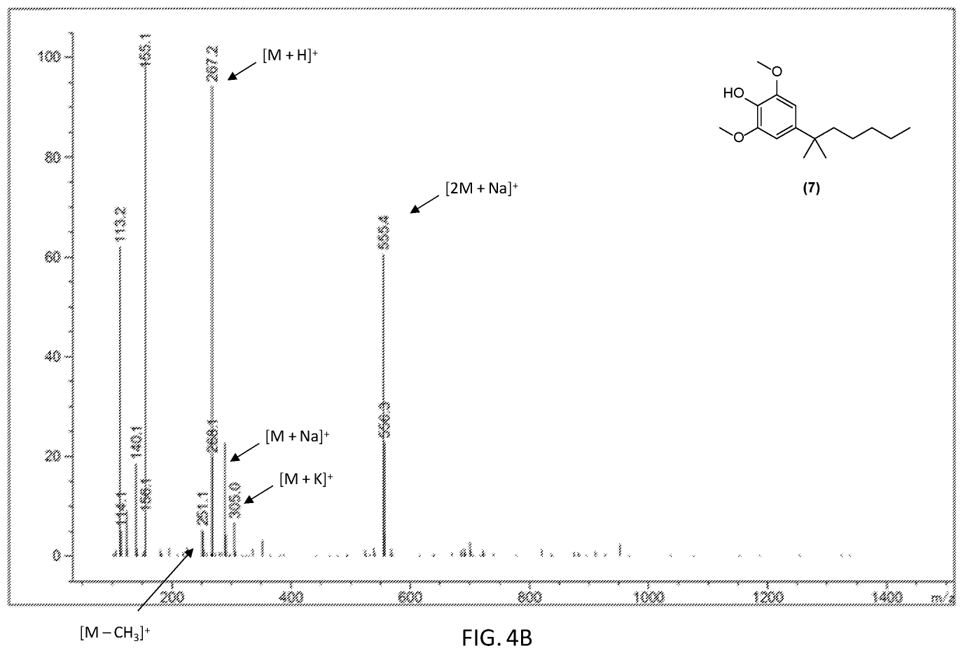

A first alkyl-chain impurity, Compound (7) (e.g., one carbon less impurity) having a relative retention time of 0.96 was characterized by ¹H NMR (FIG. 4A) and mass spectrometry (FIG. 4B) and is expected to have the following structure

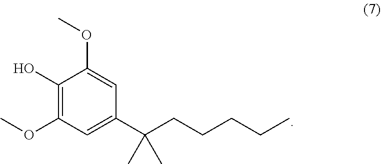 [SMILES: CCCCCC(C)(C)c1cc(OC)c(O)c(OC)c1]

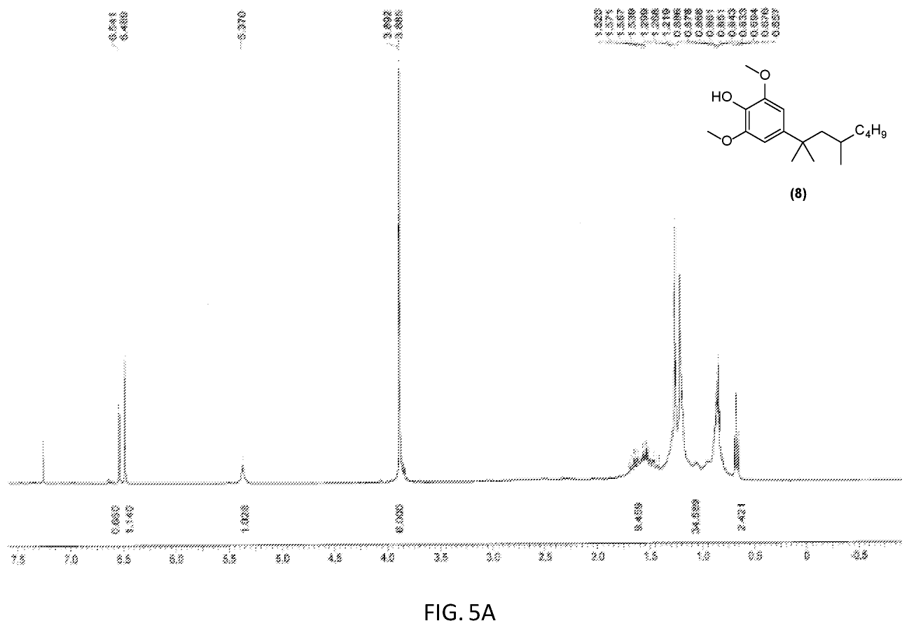

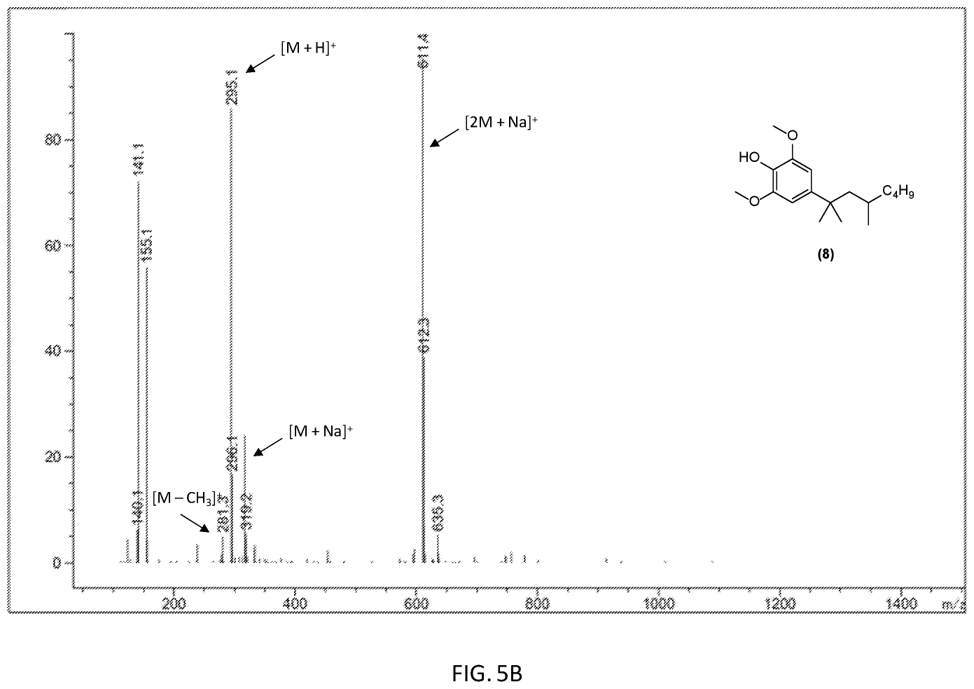

A second alkyl-chain impurity, Compound (8) (e.g., one carbon more impurity) having a relative retention time of 1.03 was characterized by ¹H NMR (FIG. 5A) and mass spectrometry (FIG. 5B) and is expected to have the following structure

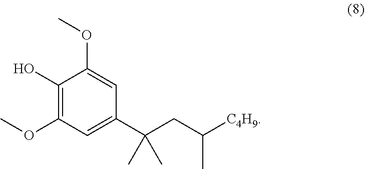

On the basis of these results, the inventors determined that Step 2 of the synthesis of DMHR, as described in Example 1, which includes the coupling of 2-methyloctan-2-ol and 1,3-dimethoxy-2-hydroxybenzene to produce compound (4) allows for the formation of homologous alkyl-chain impurities in the production of compound (4), (e.g., homologous alkyl-chain impurities having the structure of Formula (I)). A proposed mechanism for the formation of homologous alky-chain impurities in Step 2 of the synthesis of DMHR is provided in FIG. 6. The inventors further determined that impurities generated in this step may be carried through in the production of DMHR (e.g., homologous alkyl-chain impurities having the structure of Formula (II)).

### Example 5. Synthesis of Ultrapure DMHR

To minimize the presence of homologous alkyl chain impurities in the preparation of DMHR, ultrapure DMHR was synthesized according to the scheme provided in FIG. 2. The preparation of ultrapure DMHR was performed according to the synthetic protocol provided in Example 1 (e.g., the synthetic protocol for the original synthesis of DMHR), with the following modifications:

### Modifications to Synthesis of Compound (2)

2-MeTHF was used as the solvent instead of THF, providing better phase separation in the workup of Compound (2).

### Modifications to the Synthesis of Compound (4)

The reaction of 2-methyloctan-2-ol (Compound 2) with 1,3-dimethoxy-2-hydroxybenzene (Compound 3) under acidic conditions was modified according the following reaction conditions to minimize the production of homologous alkyl-chain impurities:

(i) The solution containing 2-methyloctan-2-ol was added to the acidic solution containing 1,3-dimethoxy-2-hydroxybenzene over 6 hours. The resulting decrease in the production of homologous alkyl chain impurities in the synthesis of Compound (4) is shown in FIG. 7, as compared to the production of alkyl chain impurities resulting from the original conditions which featured a more rapid addition of 2-methyloctan-2-ol to 1,3-dimethoxy-2-hydroxybenzene over 1 hour or less.

(ii) The reaction was performed with a molar excess of 1.2 equivalents of 1,3-dimethoxy-2-hydroxybenzene over 1 equivalent of 2-methyloctan-2-ol. The resulting decrease in the production of homologous alkyl chain impurities in the synthesis of Compound (4) is shown in FIG. 8, as compared to the production of alkyl chain impurities resulting from the original conditions having a 1:1.1 molar ratio of 1,3-dimethoxy-2-hydroxybenzene to 2-methyloctan-2-ol.

Two further modifications to the synthesis of Compound (4) were independently investigated for their ability to reduce the production of homologous alkyl chain impurities:

(iii) The rate of production of the Compound (4) and corresponding homologous alkyl chain impurities was determined and the production of these compounds over time is shown in FIG. 9. The more rapid initial rate of production of Compound (4) as compared to the impurities suggests that quenching the reaction at a time point prior to completion of the reaction (e.g., after 2 hours or before 75% of the 2-methyloctan-2-ol is converted into Compound (4)) may increase the ratio of product to alkyl-chain impurities.

(iv) The reaction of 2-methyloctan-2-ol and 1,3-dimethoxy-2-hydroxybenzene to produce Compound (4) was performed at a reduced temperature of 25° C. for an initial period of time, followed by an increase to 35° C. for a second period of time, and 45° C. for a third period of time. This was compared to the reaction under the original conditions of 50° C. FIG. 10 shows that reduction in reaction temperature decreases the production of homologous alkyl-chain impurities.

Finally, the production of Compound (4) was further modified by formation of a sodium salt of Compound (4), which was isolated by crystallization. Crystallization of the sodium salt further assists with removal of impurities from Compound (4).

### Modifications to the Synthesis of Compound (6)

The catalyst loading was decreased from 0.11 equivalents to 0.03 equivalents and the bulk of the MeOH was removed before the aqueous workup to increase clean phase separation.

Modifications to the Synthesis of DMHR BBr₃ loading was decreased from 2.4 equivalents to 1.65 equivalents to make subsequent quenching faster and safer. Furthermore, the BBr₃ addition temperature was increased from −78° C. to −10 to 0° C., to reduce energy consumption and enable a much broader selection of reactors. Additionally, 0 to 5° C. 1 N aqueous K₃PO₄ was used instead of the ice water quench to improve the typical assay of isolated DMHR from about 92% to greater than 97% and, in particular, to purge a t-butyl impurity (RRT 0.18) which is otherwise not removed by the water wash or crystallization. Finally, MTBE was used as the extraction solvent rather than CH₂Cl₂, significantly reducing the formation of emulsions, and the crystallization solvent system of 8 vol. of 40:1 methylcylohexane/MTBE was used instead of 102 vol. of 50:1 heptane/CH₂Cl₂.

### Example 6. Characterization of Ultrapure DMHR

Two large-scale batches of ultrapure DMHR were produced according to the synthetic scheme of FIG. 2; the corresponding synthetic methods are further described in Example 5. The batches were characterized by HPLC, as described in Example 2, the results of which are provided in Table 6. Table 6 shows that the resulting 88 kg and 89 kg batches of ultrapure DMHR each were found to have a purity of greater than 98% (99.4% and 99.5%, respectively) and decreased levels of homologous alkyl-chain impurities.

TABLE 6

| Ultrapure | | | | |
|---|---|---|---|---|
| DMHR | | | Compound (7) | Compound (8) |
| Batch Size | Assay | Purity | RRT 0.96 | RRT 1.03 |
| (kg) | (% w/w) | (% AUC) | (% AUC) | (% AUC) |
| 88 | 99.3 | 99.4 | 0.07 | 0.05 |
| 89 | 99.4 | 99.5 | 0.09 | 0.05 |

### Example 7. Synthesis of Ajulemic Acid Using Ultrapure DMHR

Synthesis of ajulemic acid using ultrapure DMHR was performed according to the following methods. The corresponding synthetic scheme is provided in FIG. 13.

To a reactor were charged ultrapure DMHR (1.22 kg, 1 equiv.), TsOH.H₂O (20.7 g, 0.02 equiv.) and toluene (6.71 kg, 7.74 L, 6.3 vol.). While maintaining the batch temperature at 33 to 37° C., a solution of PMD (865 g, 1.1 equiv.) in toluene (1.16 kg, 1.34 L, 1.1 vol.) was added over 90 mins, followed by a toluene rinse (0.37 kg, 0.42 L, 0.35 vol.). The batch was agitated for 1 hour, then heated to 50-80° C. under partial vacuum, using a Dean-Stark trap to remove water by azeotropic distillation. Once drying was complete, TsOH.H₂O (20.7 g, 0.02 equiv.) was charged and the batch agitated at 70-80° C. for 16 hours. Pyridine (543 g, 553 mL, 1.3 equiv.) was charged to the batch with a toluene (214 g, 246 mL, 0.2 vol.) rinse, followed by acetic anhydride (690 g, 638 mL, 1.3 equiv.) with a toluene (104 g, 120 mL, 0.1 vol.) rinse, each added over 30 mins while maintaining the batch temperature at 70-80° C. After 20 hours water (9.77 kg, 9.77 L, 8 vol.) was added and the batch temperature was adjusted to 50-60° C. The organic layer was further washed with two portions of water (each 2.44 kg, 2.44 L, 2.0 vol.) at 50-60° C. The batch was concentrated under reduced pressure at <80° C. to about 1.5 L and IPA (4.80 kg, 6.10 L, 5 vol. equiv.) was added. The partial concentration was repeated twice more with another IPA recharge in between. IPA (8.64 kg, 11.0 L, 9.0 vol.) was added and the batch temperature adjusted to 45-55° C. Water (3.11 kg, 3.11 L, 2.55 vol.) was added, and the batch held at temperature for an hour before cooling to 25° C. over an hour. The batch was held at this temperature for over 14 hours, then cooled to 0 to 10° C. over an hour and held there for another hour before filtering it, washing the cake with a chilled solution of water (1.59 kg, 1.59 L. 1.3 vol.) in IPA (5.00 kg, 6.35 L, 4.0 vol.). The product was dried under reduced pressure at 45-55° C. to afford 1.54 kg of Compound (13) (99.53% purity by HPLC, 77% yield).

Compound (13) (1.50 kg, 1 equiv.), selenium dioxide (403 g, 1.25 equiv.), tetrahydrofuran (6.13 kg, 6.90 L, 4.6 vol.) and water (300 g, 300 mL, 0.2 vol.) were charged to a reactor. The batch was heated at 55-55° C. for 21 hours, then cooled to 0-10° C. and maintained there while 35 wt % hydrogen peroxide (1.42 kg, 1.26 L, 3.0 equiv.) was charged slowly. The batch was agitated for another 30 mins., then warmed to 20-25° C. and held there for another 13 hours, before slowly quenching it with 20 wt % aqueous sodium thiosulfate (4.6 kg, 3.9 L, 2.6 vol., 2 equiv.) at <35° C. After 4 hours at 15-25° C., no peroxide was detected in the mixture. The batch was filtered through a pad of Celite, rinsing with THE (1.34 kg, 1.50 L, 1 vol.). The filtrate's organic layer was washed with 10 wt % sodium chloride (3.0 kg, 2.8 L, 1.9 vol.), then cooled to below 5° C. and held there while 30 wt % aqueous sodium hydroxide (969 g, 730 mL, 2.0 equiv.) was charged. The batch was warmed to 25-25° C. for 15 hours, then diluted with heptane (1.0 kg, 1.5 L, 1.0 vol.), water (2.4 kg, 2.4 L, 1.6 vol.) and toluene (4.5 kg, 5.2 L, 3.5 vol.) at 15-25° C. The aqueous layer was discarded and the organic layer diluted with heptane (5.0 kg, 7.2 L, 4.8 vol.) and water (4.7 kg, 4.7 L, 3.1 vol.); the resulting organic layer was discarded. The aqueous layer was extracted twice with heptane (4.9 kg, 7.1 L, 4.75 vol.), adding THE (670 g, 750 mL, 0.5 vol.) to the second extraction. MTBE (6.3 kg, 8.5 L, 5.7 vol.) was charged to the aqueous layer and the batch cooled to −5 to 5° C. before acidifying to <pH 1.5 using 35 wt % hydrochloric acid. The aqueous phase was discarded and the organic phase washed with 10 wt % sodium chloride (3.0 kg, 2.8 L, 1.9 vol.) The organic phase was concentrated at 30-60° C. under reduced pressure to about 2 L, after which acetonitrile (2.3 kg, 3.0 L, 2.0 vol.) was added. The partial concentration/acetonitrile recharge sequence was repeated twice more before diluting the batch up to 3.0 L (2.0 vol.) with acetonitrile, adjusting its temperature to 45-50° C. and cooling it to 23-27° C. over at least three hours. The batch was held at that temperature for another 3 hours, then further cooled to 3-7° C. over at least another 3 hours and held there for 3-4 hours before filtering, washing the cake with cold acetonitrile (1.2 kg, 1.5 L, 1.0 vol.). The product was dried under reduced pressure at 45-55° C. to yield 307 g of Compound (16) (crude ajulemic acid; 98.63% purity by HPLC, 20% yield).

Heptane (1.5 kg, 2.2 L, 9 vol.) and pyridine (104 g, 106 mL, 2.1 equiv.) were charged to a reactor and the solution heated to 50 to 60° C. Compound (16) (250 g, 1.0 equiv.) was added, followed by acetic anhydride (115 g, 106 mL, 1.8 equiv.). After 4 hours, water (350 g, 350 mL, 1.4 vol.) was added slowly while maintaining the temperature at 50-60° C.; the batch was stirred for 22 hours at the same temperature. The aqueous layer was discarded and the organic layer washed with 6 wt % aqueous H₃PO4 (648 g, 700 mL, 2.8 vol.), then twice with water (350 g, 350 mL, 1.4 vol.). The batch was cooled to 20-30° C. over at least two hours, then to 0-5° C. over at least another two hours, and finally held at 0-5° C. for three hours before filtering. The cake was washed with cold heptane (340 g, 500 mL, 2 vol.) and dried at 45-55° C. under reduced pressure to afford 238 g of Compound (17) (99.29% purity by HPLC, 86.2% yield).

Compound (17) (169 g, 1 equiv.) and MTBE (516 g, 698 mL, 4.13 vol. equiv.) were charged to a reactor and 2N NaOH (493 g, 456 mL, 2.7 vol.) was added while maintaining the batch temperature below 50° C. The batch temperature was held at 45-55° C. for 1.5 hours, then reduced to 20-30° C. and maintained there while the batch was acidified with 37 wt % hydrochloric acid (120 g, 101 mL, 0.60 vol.). The organic layer was washed with water (186 g, 186 mL, 1.1 vol.), then filtered through Celite, rinsing with MTBE (63 g, 85 mL, 0.5 vol. equiv.). The filtrate was partially concentrated under reduced pressure at 15-25° C., removing 340 mL (2 vol.) of distillate. Acetonitrile (534 g, 680 mL, 4 vol.) was added and additional solvent (680 mL, 4 vol.) distilled off before repeating the acetonitrile addition and partial concentration again. The batch temperature was adjusted to 20-30° C., then reduced to −10 to −5° C. over at least 2 hours, where it was held for 3 hours before filtering the batch. The cake was washed with cold acetonitrile (267 g, 340 mL, 2 vol.), then dried under reduced pressure at 50-55° C. to provide 143 g of ajulemic acid (JBT-101; 99.71% purity by HPLC, 93% yield).

### Example 8. Analysis of Ajulemic Acid Synthesized Using Ultrapure DMHR

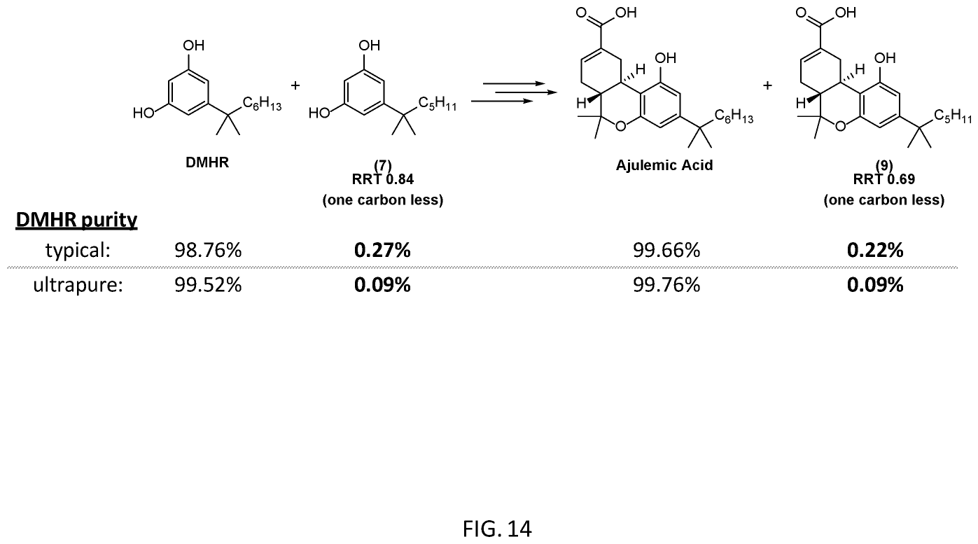

Ajulemic acid, and synthetic intermediates in the production of ajulemic acid, were characterized according to the general methods described below. Ajulemic acid was synthesized using ultrapure DMHR as a starting material as described in Example 7. The resulting ajulemic acia was assayea Dy HPLC as described herein and determined to have a purity of 99.76% and 0.09% of homologous alkyl chain impurity (RRT 0.84, one carbon less). This is in contrast to ajulemic acid produced using conventional DMHR, which had a purity of 99.66% and 0.22% of the particular homologous alkyl chain impurity (RRT 0.69, one carbon less), representing a greater than two-fold increase in the impurity in ajulemic acid (FIG. 14).

### IR Spectrum

The infrared spectrum of ajulemic acid (JBT-101) was recorded on a neat sample using a Nicolet iS50 spectrometer with an attenuated total reflectance probe.

### ¹H-NMR Spectrum

The ¹H-NMR spectrum of ajulemic acid (JBT-101) was obtained on a sample that was dissolved in CDCl₃ taken on a 400 MHz Bruker Advance Shield Spectrometer.

### ¹³C-NMR Spectrum

The ¹³C-NMR spectrum of ajulemic acid (JBT-101) was obtained on a sample dissolved in CDCl₃ taken on a 400 MHz Bruker Advance Spectometer

### Stereochemistry

The chiral purity of ajulemic acid (JBT-101) was obtained by isocratic elution high performance liquid chromatography (HPLC) using a Phenomenex Lux 5p Cellulose-1, 250×4.6 mm chiral column. The mobile phase was a mixture of heptane, ethanol and trifluoroacetic acid at a ratio of 94.9:5:0.1 volume to volume (v/v) respectively. The detection wavelength was set to 225 nm. The enantiomer of JBT-101 was used as an external standard to perform quantitation.

### X-Ray Powder Diffraction

The X-Ray Powder Diffraction (XRPD) of ajulemic acid (JBT-101) was obtained on a Bruker D2 Phaser X-Ray diffractometer using CuKα radiation at 30 kV, 10 mA, over a range of 4.0-50 degrees 2θ.

### Assay and Impurities Quantitation

The assay and organic impurities of ajulemic acid (JBT-101) were quantitated by gradient elution HPLC using an Agilent Eclipse XBD C8, 150×4.6 mm, 5 μm particle size. Mobile phase A was a mixture of water, acetonitrile, and phosphoric acid at a ratio of 45:55:0.1 (v/v) respectively. Mobile phase B was a mixture of acetonitrile and phosphoric acid at a ratio of 100:0.1 (v/v) respectively. The gradient details are outlined below in Table 7. The detection wavelength was set to 230 nm. A qualified reference standard of JBT-101 was used as an external standard to perform quantitation of the main band and impurities.

TABLE 7

| Time (min) | % Mobile Phase A | % Mobile Phase B |
|---|---|---|
| 0.0 | 100 | 0 |
| 3.0 | 100 | 0 |
| 45.0 | 95 | 5 |
| 50.0 | 5 | 95 |
| 50.5 | 100 | 0 |
| 55.0 | 100 | 0 |

### Water Content

The water content of ajulemic acid (JBT-101) was determined by coulometric titration of a Metrohm 756 Coulometer. A sample of ajulemic acid is added to a vessel containing anhydrous methanol. The solution is titrated with Hydranal® Coulomat solution which generates free iodine in solution via an electrochemical reaction. The electrical current passed through the solution is inversely proportional to the amount of iodine in solution. The endpoint of the titration is determined voltrametrically by applying an alternating current of constant strength to a double Pt electrode that is submerged in the solution. The voltage difference decreases drastically when traces of iodine are present, indicating the endpoint. The water content is calculated directly from the amount of coulomat that is titrated to reach the endpoint. An external standard of Hydranal® Water standard is run to confirm the system is accurate.

### Residue on Ignition (ROI)

The residue on ignition was performed using the sulfated ash method described in USP <281>. A sample of ajulemic acid (JBT-101) was ignited in a crucible in the presence of sulphuric acid until white fumes are no longer visible. The amount of residue remaining after ignition was calculated by differential weighing.

### Elemental Impurity Analysis

Inductively Coupled Plasma Mass Spectrometry (ICP-MS) was performed to determine the concentration of the following elemental impurities in ajulemic acid (JBT-101): Selenium, Cadmium, Lead, Arsenic and Mercury. Quantitation was performed against external standards of each element.

### Residual Solvents

The concentration of residual solvents in ajulemic acid (JBT-101) was quantitated using an Agilent Gas Chromatograph (GC) configured with a headspace autosampler and an Agilent J&W DB624 capillary column, 30 m (L)×0.32 mm (ID)×1.8 μm (df). A vial pressure of 15 psi was applied to the 10 mL sample vials with a sample oven temperature of 100° C. A 1.0 min injection was performed on a 225° C. inlet with a 2 mm deactivated liner and a 5:1 split flow. The oven gradient that the column was subjected to during separation is defined below in Table 8. Helium was used as the carrier gas with a constant flow rate of 1.5 mL/min. Detection was performed using a Flame Ionization Detector (FID) operating at 270° C.

The following solvents were quantitated against external stanaaras using the method described above: acetonitrile, tetrahydrofuran, toluene, acetone, propan-2-ol, n-heptane, methyl tert butyl ether, and tert butanol.

TABLE 8

| Ramp (° C./min) | Hold Time (min) | Final Temperature (° C.) |
|---|---|---|
| N/A | 4 | 40 |
| 8 | 0 | 60 |
| 5 | 2 | 85 |
| 30 | 2 | 220 |

### Pyridine

The concentration of pyridine in ajulemic acid (JBT-101) was quantitated by performing direction injection of sample on an Agilent Gas Chromatograph 6890 (GC) and an Agilent J&W DB624 capillary column, 30 m (L)×0.32 mm (ID)×1.8 μm (df). A 1 μL injection was performed through a 250° C. inlet with a 3:1 split flow. The oven gradient that the column was subjected to during separation is defined below in Table 9. Helium was used as the carrier gas with a constant pressure of 4 psi. Detection was performed using a Flame Ionization Detector (FID) operating at 300° C. The concentration of pyridine in the sample was quantitated against an external standard of pyridine.

TABLE 9

| Ramp (° C./min) | Hold Time (min) | Final Temperature (° C.) |
|---|---|---|
| N/A | 5 | 40 |
| 10 | 3 | 260 |

### Benzene

The concentration of benzene in ajulemic acid (JBT-101) was quantitated using an Agilent Gas Chromatograph (GC) configured a headspace auto sampler and an Agilent J&W DB624 capillary column, 30m (L)×0.32 mm (ID)×1.8 μm (df). A vial pressure of 14 psi was applied to the 10 mL sample vials with a sample oven temperature of 85° C. A 1.0 min inject time was performed on 200° C. inlet with a 1:1 split flow. The oven gradient that the column was subjected to during separation is defined below in Table 10. Helium was used as the carrier gas with a constant flow rate of 3.5 mL/min. Detection was performed using a Flame Ionization Detector (FID) operating at 300° C. The concentration of benzene in the sample was quantitated against an external standard of benzene.

TABLE 10

| Ramp (° C./min) | Hold Time (min) | Final Temperature (° C.) |
|---|---|---|
| N/A | 0 | 40 |
| 2 | 0 | 70 |
| 35 | 3 | 245 |

### Differential Scanning Calorimetry (DSC)

Differential scanning calorimetry was performed on ajulemic acid (JBT-101) sample by applying a temperature gradient of 10.00° C./min over a range of 30.0-350.0° C. The difference in the amount of heat required to increase the temperature of the sample as compared to a reference is measured as a function of temperature.

### Particle Size Distribution (PSD)

The particle size distribution of ajulemic acid (JBT-101) was measured using a wet dispersion method on a Malvern Mastersizer 3000. A sample of ajulemic acid powder was added to a dilute Tween 80 solution in water then sonicated for 1 min to break up agglomerates. The dispersion units' speed was set to 1700 rpm and an obscuration of 10-30%. A Fraunhofer scattering model was applied to the data.

### Ajulemic Acid Impurities Quantitation by HPLC

The assay and organic impurities of ajulemic acid (JBT-101) were quantitated by gradient elution HPLC using an Agilent Eclipse XBD C8, 150×4.6 mm, 5 μm particle size. Mobile phase A was a mixture of water, acetonitrile, and phosphoric acid at a ratio of 45:55:0.1 (v/v/v) respectively. Mobile phase B was a mixture of acetonitrile and phosphoric acid at a ratio of 100:0.1 (v/v) respectively. The flow rate was set to 1.0 mL/min. The gradient details are outline below in Table 11. The detection wavelength was set to 230 nm. A qualified reference standard of ajulemic acid was used as an external standard to perform quantitation of the main band and impurities.

TABLE 11

| Time (min) | % Mobile Phase A | % Mobile Phase B |
|---|---|---|
| 0.0 | 100 | 0 |
| 3.0 | 100 | 0 |
| 45.0 | 95 | 5 |
| 50.0 | 5 | 95 |
| 50.5 | 100 | 0 |
| 55.0 | 100 | 0 |

### Ajulemic Acid Intermediates, Impurities Quantitation by HPLC, Method 1

The assay and organic impurities of ajulemic acid (JBT-101) intermediates were quantitated by gradient elution HPLC using an Phenomenex Kinetic F5, (150 mm×4.6 mm, 2.6 μm particle size). Mobile phase A was 0.1% trifluoroacetic acid in water. Mobile phase B was 0.1% trifluoroacetic acid in acetonitrile. The flow rate was set to 1.2 mL/min. The gradient details are outline below in Table 12. The detection wavelength was set to 230 nm. Impurities were calculating by determining the percent area of each impurity as compared to the total chromatographic area.

TABLE 12

| Time (min) | % Mobile Phase A | % Mobile Phase B |
|---|---|---|
| 0.0 | 55 | 45 |
| 35.0 | 30 | 70 |
| 40.0 | 30 | 70 |

### Ajulemic Acid Intermediates, Impurities Quantitation by HPLC, Method 2

The assay and organic impurities of ajulemic acid (JBT-101) intermediates were quantitated by gradient elution HPLC using an Phenomenex Kinetic F5, (150 mm×4.6 mm, 2.6 μm particle size). Mobile phase A was 0.05% trifluoroacetic acid in water. Mobile phase B was 0.05% trifluoroacetic acid in acetonitrile. The flow rate was set to 0.7 mL/min. The gradient details are outline below in Table 13. The detection wavelength was set to 230 nm. Impurities were calculating by determining the percent area of each impurity as compared to the total chromatographic area.

TABLE 13

| Time (min) | % Mobile Phase A | % Mobile Phase B |
|---|---|---|
| 0.0 | 35 | 65 |
| 35.0 | 0 | 100 |
| 32.0 | 0 | 100 |

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

What is claimed is:

1. A method of making compound (4): 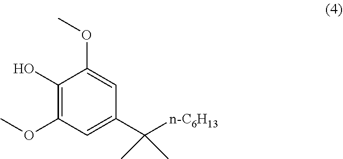
wherein said method comprises the step of:
(i) adding a solution containing 1 molar equivalent of 2-methyloctan-2-ol to an acidic solution containing at least 1.1 molar equivalents of 1,3-dimethoxy-2-hydroxybenzene to form a mixture of compounds of formula (I): 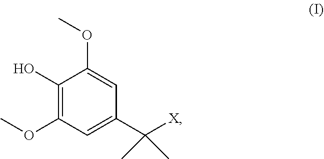
wherein X is a linear or branched C1-C10 alkyl, and wherein the mixture comprises at least 98.0% compound (4) and less than 2.0% compounds of formula (I) in which X is a linear or branched C1-C10 alkyl other than n-C₆H₁₃,
wherein the solution containing 2-methyloctan-2-ol is added to the acidic solution containing 1,3-dimethoxy-2-hydroxybenzene over the course of at least 1 hour; or
wherein step (i) is performed at a temperature of between 20° C. and 55° C.

2. The method of claim 1, wherein step (i) comprises adding a solution containing 1 molar equivalent of 2-methyloctan-2-ol to an acidic solution containing at least 1.2 molar equivalents of 1,3-dimethoxy-2-hydroxybenzene.

3. The method of claim 1, wherein the solution containing 2-methyloctan-2-ol is added to the acidic solution containing 1,3-dimethoxy-2-hydroxybenzene over the course of at least 2 hours.

4. The method of claim 3, wherein the solution containing 2-methyloctan-2-ol is added to the acidic solution containing 1,3-dimethoxy-2-hydroxybenzene over the course of at least 4 hours.

5. The method of claim 3, wherein the solution containing 2-methyloctan-2-ol is added to the acidic solution containing 1,3-dimethoxy-2-hydroxybenzene over the course of at least 6 hours.

6. The method of any one of claims 1-3, wherein step (i) is quenched ater 2 hours.

7. The method of any one of claims 1-3, wherein step (i) is quenched before 75% of the 2-methyloctan-2-ol is converted into compound (4).

8. The method of claim 7, wherein step (i) is quenched before 65% of the 2-methyloctan-2-ol is converted into compound (4).

9. The method of any one of claims 1-7, wherein step (i) is performed at a temperature of between 20° C. and 50° C.

10. The method of claim 9, wherein step (i) is performed at a temperature of between 30° C. and 45° C.

11. The method of any one of claims 1-10, wherein the mixture comprises at least 99.0% compound (4).

12. The method of claim 11, wherein the mixture comprises at least 99.5% compound (4).

13. The method of anyone of claims 1-12, wherein the mixture comprises less than 0.75% compounds of formula (I) in which X is a linear or branched C1-C10 alkyl other than n-C₆H₁₃.

14. The method of claim 13, wherein the mixture comprises less than 0.25% compounds of formula (I) in which X is n-C₅H₁₁ or —CH₂CH(CH₃)CH₂CH₂CH₂CH₃.

15. The method of claim 14, wherein the mixture comprises less than 0.15% compounds of formula (I) in which X is n-C₅H₁₁ or —CH₂CH(CH₃)CH₂CH₂CH₂CH₃.

16. The method of anyone of claims 1-15, wherein step (1) comprises producing greater than 0.5 kg of compound (4).

17. The method of claim 16, wherein step (1) comprises producing greater than 5 kg of compound (4).

18. The method of any one of claims 1-17, further comprising subjecting compound (4) to hydrogenation and demethylation to produce 5-(1,1-dimethylheptyl)resorcinol (DMHR): 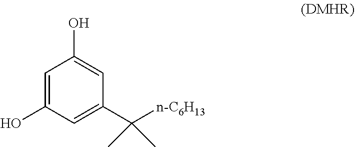
in a mixture of compounds of formula (II): 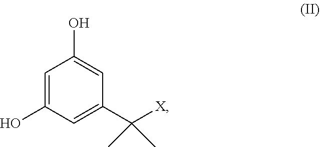
wherein X is a linear or branched C1-C10 alkyl, and wherein the mixture comprises at least 98.0% DMHR and less than 2.0% compounds of formula (II) in which X is a linear or branched C1-C10 alkyl other than n-C₆H₁₃.

19. The method of claim 18, further comprising reacting para-mentha-2,8-dien1-ol (PMD) and DMHR to form compound (12): 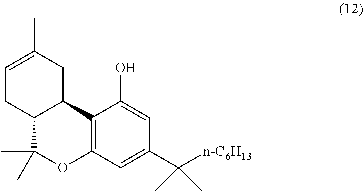
in a mixture of compounds of formula (III): 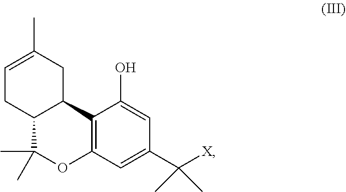
wherein X is a linear or branched C1-C10 alkyl, and wherein the mixture comprises at least 98.0% compound (12) and less than 2.0% compounds of formula (III) in which X is a linear or branched C1-C10 alkyl other than n-C₆H₁₃.

20. The method of claim 19, further comprising oxidizing compound (12) to form ajulemic acid (AJA): 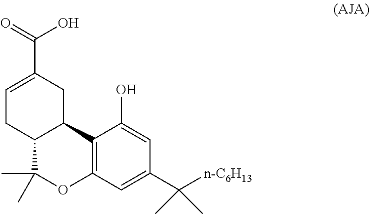
in a mixture of compounds of formula (IV): 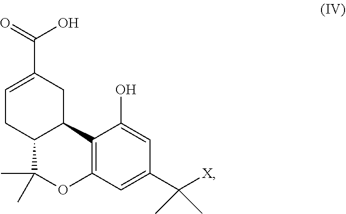
wherein X is a linear or branched C1-C10 alkyl, and wherein the mixture comprises at least 98.0% AJA and less than 2.0% compounds of formula (IV) in which X is a linear or branched C1-C10 alkyl other than n-C₆H₁₃.

21. A pharmaceutical composition comprising ajulemic acid, or a salt thereof, produced according to the method of claim 19 and a pharmaceutically acceptable excipient.

22. A method of treating an inflammatory condition in a subject in need thereof, the method comprising administering to the subject the pharmaceutical composition of claim 21 in an amount sufficient to treat the condition.

23. A method of treating a fibrotic condition in a subject in need thereof, the method comprising administering to the subject the pharmaceutical composition of claim 21 in an amount sufficient to treat the condition.

24. The method of claim 18, further comprising reacting 5-(1,1-dimethylheptyl)resorcinol (DMHR) to produce a cannabinoid.

25. The method of claim 24, wherein the cannabinoid is a dimethylheptyl-cannabidiol analog.

26. The method of claim 25, wherein the dimethylheptyl-cannabidiol analog is selected from any one of Compounds 20-53.

27. The method of claim 24, wherein the cannabinoid is a dimethylheptyl-tetrahydrocannabinol analog.

28. The method of claim 25, wherein the dimethylheptyl-tetrahydrocannabinol analog is selected from any one of Compounds 54-125.

29. The method of claim 24, wherein the cannabinoid is ajulemic acid.

30. A pharmaceutical composition comprising a cannabinoid, or a salt thereof, produced according to the method of any one of claims 24-29 and a pharmaceutically acceptable excipient.
